(19) United States
(12) Patent Application Publication
(10) Pub. No.: US 2026/0250636 A1
(43) Pub. Date: Aug. 27, 2026

(54) A METHOD FOR PRODUCING A BIOENGINEERED MAMMAL INDUCED PLURIPOTENT STEM CELL-DERIVED CARDIAC ORGANOID

(71) Applicants: INSTITUT NATIONAL DE LA SANTÉ ET DE LA RECHERCHE MÉDICALE, Paris (FR); UNIVERSITE PARIS CITE, Paris (FR); UNIVERSITE PARIS-SACLAY, Gif-sur-Yvette (FR); ASSISTANCE PUBLIQUE - HOPITAUX DE PARIS (APHP), Paris (FR); INSTITUT GUSTAVE ROUSSY, Villejuif (FR)

(72) Inventors: Ali TURHAN, Villejuif (FR); Annelise BEANNACEUR GRISCELLI, Villejuif (FR); Franck GRISCELLI, Villejuif (FR); Jinwook HWANG, Villejuif (FR)

(21) Appl. No.: 18/874,976

(22) PCT Filed: Jun. 20, 2023
(86) PCT No.: PCT/EP2023/066629

§ 371 Date: Dec. 13, 2024

(30) Foreign Application Priority Data
Jun. 21, 2022 (EP) 22305899.1

(51) Int. Cl. C12N 5/077; G01N 33/50

(52) U.S. Cl. CPC C12N 5/0657; G01N 33/5061; C12N 2501/12; C12N 2503/02; C12N 2506/45; C12N 2513/00

(57) ABSTRACT

Cardiovascular diseases represent the leading cause of mortality and accounts for approximately one-third of deaths globally each year. During the last decade, tremendous efforts have been made to take advantage of advances in regenerative cardiology using the approaches of adult or pluripotent stem cells. However, the current protocols do not recapitulate cardiac-related architectures. Thus, there remains a need in the art for further methods for producing cardiac organoids actually mimicking a cardiac tissue, starting from pluripotent stem cells. Notably, there remains a need in the art for producing cardiac organoids comprising cardiac muscle cells and endothelial cells, including both atrium-like and ventricular-like chambers. Such cardiac organoids that reliably mimic a cardiac tissue would notably allow testing for the physiological effects of known drug substances as well as candidate molecules, particularly in embodiments wherein the said cardiac organoids are produced by starting from cells originating from patients affected with a cardiac disease or disorder such as a cardiac disease or disorder of genetic origin. It is now provided a novel matrix-free and feeder-free method of generating mammal iPSC-derived cardiac organoids, which method includes both steps of dynamic culture conditions and static culture conditions. As it is experimentally shown, cardiac organoids obtained with the matrix-free and feeder-free method disclosed herein are relevant models for testing potentially active substances on cardiac organoids obtained from iPSCs derived from subjects affected with a cardiac disease or disorder.

### BACKGROUND OF THE INVENTION

Cardiovascular diseases represent the leading cause of mortality and accounts for approximately one-third of deaths globally each year. During the last decade, tremendous efforts have been made to take advantage of advances in regenerative cardiology using the approaches of adult or pluripotent stem cells. The revolution of iPSC technology generated a novel burst of interests in this field by the ability of creating in vitro, all types of differentiated cells, including cardiomyocytes (Lee et al., 2016, Circulation, Vol. 133:2618-26323; Polanco et al., 2020, Trends in Biotechnology, Vol. 38 (10):1128-1140; Chen et al., 2015, Cell Research, Vol. 15:365-375). More recently, the organoid technology to create organ-like structures in vitro, generated a major enthusiasm in regenerative medicine and oncology. As opposed to their use in the other fields, organoid models for studying cardiac morphology, physiology and pathophysiology of cardiovascular disease have been limited so far. Cardiac organoids which have been shown previously to be generated from embryonic stem cells, can also be generated readily from both human and mouse iPSC (Lee et al., 2020, Nat. Commun., Vol. 11:4283; Drakhlis et al., 2021, Nat. Biotechnol., Vol. 39:737-746; Lewis-Israeli et al., 2021, Nat. Commun., Vol. 12:5142). Several methods have been described in the past to induce cardiomyocyte differentiation from iPSC in 2D/3D conditions (Sahara et al., 2015, EMBO J, Vol. 34:710-738; Kattman et al., 2011, Cell Stem Cell, Vol. 8:228-240; Burridge et al., Nat Methods, Vol. 11:855-860). To generate 3D cardiac organoids, the conventional methods involved several steps as well as the use of a matrix, and that of defined factors (Lee et al., 2020, Nat. Commun., Vol. 11:4283; Shkumatov et al., 2014, PLOS ONE, Vol. 9: doi:10.1371/journal.pone.0094764; Hoang et al., 2018, Nat Protocol, Vol. 13:723-737). Current methods applied to iPSC lines allow generation of 3D cardiac organoids in approximately 2-3 weeks. This organogenesis procedure requires optimally timed, sequential chemical treatments with the use of a scaffold or hydrogel. Also, the impact of hydrodynamics culture conditions have been studies (Correia et al., 2014, Stem Cell Rev and Rep, Vol. 10:786-801; Smaras et al., 2018, Journal of Biotechnology, Vol. 287:18-27). Despite their potential for heart muscle engineering, cardiac micro-tissues and heart-forming organoids, the current protocols do not recapitulate cardiac-related architectures. A recent report in this field described the generation of a left ventricle chamber model using human iPSC aggregates (Hofbauer et al., 2021, Cell, Vol. 184:3299-3317). Despite these important progresses, there are difficulties in inducing from human iPSC, a self-organizing, complex cardiac atrium- and ventricular-like structures.

In pathological situations, generation of cardiac tissues for the study of iPSC-derived cardiomyopathies has been previously explored (Ho et al., 2011, Aging, Vol. 3:380-390; Sun et al, 2012, Sci Transl Med, Vol. 4:130ra47; Thomas et al., 2022, Circulation Research, Vol. 130:1780-1802). In the case of a patient with dilated cardiomyopathy and cardiac Troponin T gene (TNNT2) mutation, patient-derived iPSCs revealed abnormalities upon differentiation towards fibroblasts and cardiomyocytes (Sun et al., 2012—Supra). Similarly, in another study of dilated cardiomyopathies in patients with laminopathies, patient-derived iPSC lines revealed nuclear abnormalities inherent to these pathologies but only upon differentiation of iPSC towards fibroblasts (Ho et al., 2011—Supra).

However, there remains a need in the art for further methods for producing cardiac organoids actually mimicking a cardiac tissue, starting from pluripotent stem cells.

Notably, there remains a need in the art for producing cardiac organoids comprising cardiac muscle cells and endothelial cells, including both atrium-like and ventricular-like chambers.

Such cardiac organoids that reliably mimic a cardiac tissue would notably allow testing for the physiological effects of known drug substances as well as candidate molecules, particularly in embodiments wherein the said cardiac organoids are produced by starting from cells originating from patients affected with a cardiac disease or disorder such as a cardiac disease or disorder of genetic origin.

### SUMMARY OF THE INVENTION

The present disclosure relates to an in vitro matrix-free and feeder-free method for producing a cardiac organoid comprising the steps of:
a) providing mammal iPSCs,
b) culturing the mammal iPSCs provided at step a) under dynamic culture conditions during a time period ranging from 30 minutes to 3 hours, whereby formation of iPSCs cell aggregates is initiated,
c) culturing under static culture conditions the cell aggregates under formation obtained at step b), during a time period ranging from 12 hours to 48 hours, whereby iPSCs cell aggregates are obtained,
d) incubating the cell aggregates obtained at step c) in a cardiomyocyte differentiation medium, whereby a differentiated myocyte-containing organoid is obtained, and
e) culturing the differentiated myocyte-containing organoid obtained at step d), whereby a cardiac organoid is obtained.

In some embodiments of the said in vitro method, the mammal iPSCs provided at step a) consist of human iPSCs.

In some embodiments of the said in vitro method, step b) is performed during a time period of about 1 hour.

In some embodiments of the said in vitro method, step d) is performed during a time period ranging from 1 to 5 days.

In some embodiments of the said in vitro method, step d) is performed by incubating the iPSCs aggregates obtained at step c) successively in more than one cardiomyocyte differentiation medium.

In some embodiments of the said in vitro method, step e) of culturing the differentiated myocyte-containing organoid obtained at step d) has a duration of 5 days or more, preferably of 7 days or more.

In some embodiments of the said in vitro method, step e) is performed during a time period ranging from 5 to 200 days.

In some embodiments of the said in vitro method, the mammal iPSCs provided at step a) derive from the programmation of a sample of differentiated human cells.

In some embodiments of the said in vitro method, the mammal iPSCs provided at step a) derive from the programmation of a sample of differentiated human cells originating from a subject affected with a cardiac disease.

The present disclosure also relates to a cardiac organoid obtainable by the in vitro method disclosed herein.

The present disclosure also pertains to the in vitro use of a cardiac organoid as disclosed herein for testing the activity of a substance endowed with physiological effects. In some embodiments, the substance endowed with physiological effects consists of a drug candidate.

The presence disclosure also concerns the in vitro use of a cardiac organoid obtainable by the method disclosed herein, wherein the mammal iPSCs derive from the programmation of a sample of differentiated human cells originating from a subject affected with a cardiac hypertrophy, for testing the activity of a drag candidate against cardiac hypertrophy. In some embodiments, the differentiated human cells have a mutation on the c-met gene.

The present disclosure also relates et a cardiac organoid having one or more of the following features:
the presence of both atrial-like and ventricular-like cell pattern,
the presence of a network of vascular capillaries, and
   the presence of contractile properties

### DESCRIPTION OF THE FIGURES

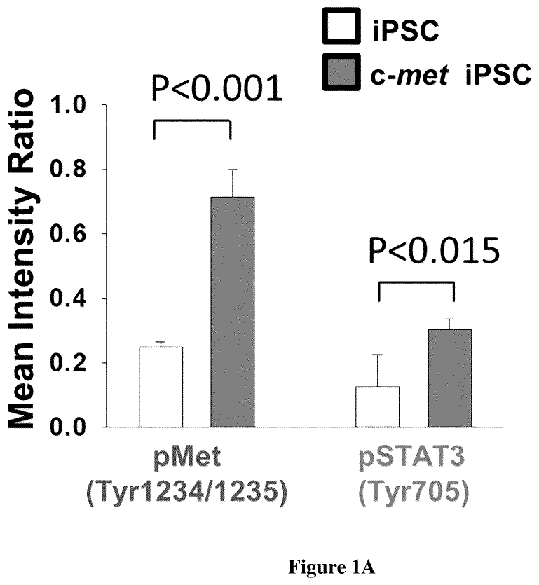

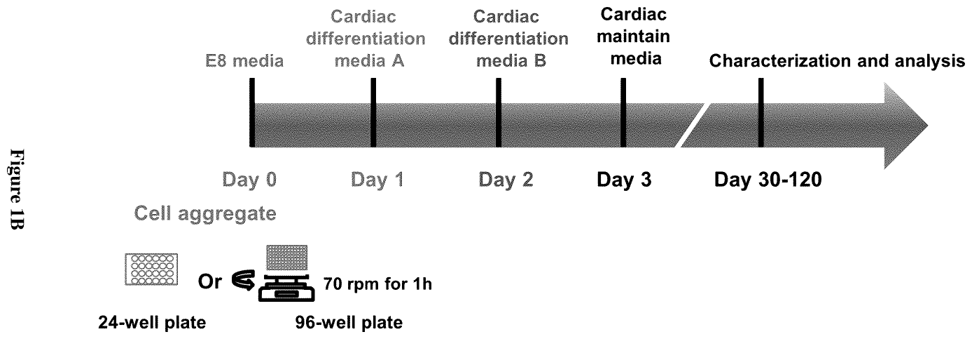

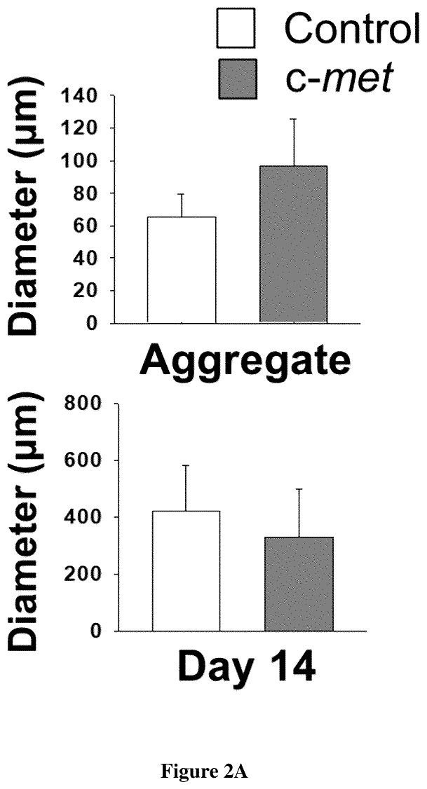

### FIG. 1

FIG. 1A: The graph shows the mean Phospho-Met (Tyr1234/1235) and Phospho-STAT3 (Tyr705) fluorescence intensity quantified (n=4).

In control (empty bars): pMet+ cells intensity; 0.24±0.01, pSTAT3+ cells intensity; 0.12±0.09.

In c-met (filled bars): pMet+ cells intensity; 0.71±0.08, pSTAT3+ cells intensity; 0.30±0.03.

Data normalized by DAPI fluorescence intensity. Data are mean±s.e.m. two-tailed Student's t-test.

Ordinate: Mean intensity ratio

FIG. 1 B: Protocol: schematic of matrix free & aggregate mediated 3D cardiac organogenesis method.

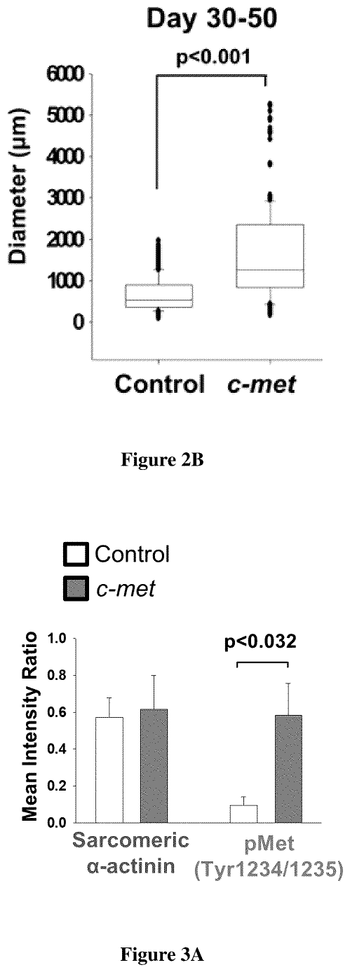

### FIG. 2

FIG. 2A: Diameter of iPSC aggregates and cardiac organoids (N=3 independent experiments). The number of organoids analyzed (n) and the number of experiments (N).

Diameter of iPSC aggregates (upper part) at 1 day (control-empty bar; n=183, c-met (filled bar; n=133),

Diameter of cardiac organoids (mower part) at 14 (control; (empty bar) n=26, c-met (filled bar); n=28), 30-50 (control; n=73, c-met; n=31) days. The Figure shows the mean size of control iPSC cardiac organoids (empty squares) as compared to c-met iPSC cardiac organoids (filled squares).

In control: aggregates: day +1, average size; 65.4±13.6 μm, control iPSC cardiac organoids: day +14, average size; 418.2±158.7 μm, and day +30-50, average size; 659.3±387.5 μm.

In c-met: aggregates: day +1, average size; 96.4±28.7 μm, c-met iPSC cardiac organoids: day +14, average size; 326.3±166.7 μm, and day +30-50, average size; 1613.7±1123.3 μm.

FIG. 2B: Boxplot of organoid diameter distribution at 30-50 days, boxplot show the minimum, first quartile, median, third quartile, and maximum, box plot indicating that c-met cardiac organoids (median; 1262.3 μm; min 175.1 μm; max 5266.9 μm) had bigger median diameters than control (median; 540.0 μm; min 101.2 μm; max 1964.8 μm). All data are mean±s.e.m. two-tailed Student's t-test.

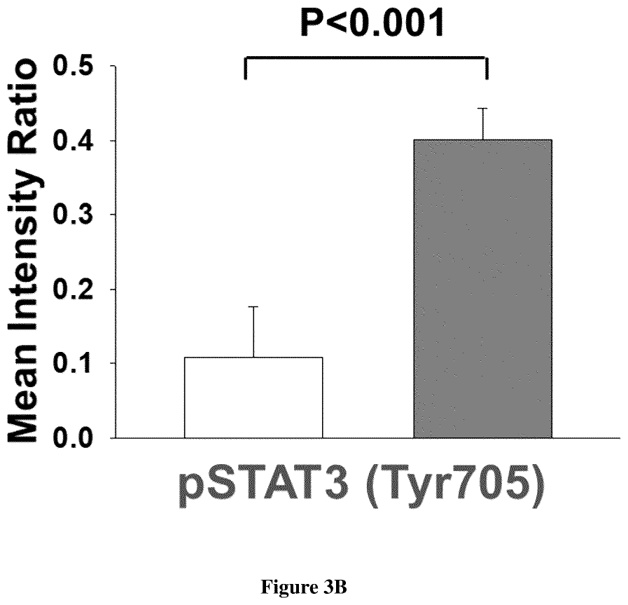

### FIG. 3

FIGS. 3A,3B: The graph shows the mean Sarcomeric Alpha Actinin, Phospho-Met (FIG. 3A) and Phospho-STAT3 (FIG. 3B) fluorescence intensity quantified (n=3), Data normalized by DAPI fluorescence intensity at 21-30 days. Data are mean±s.e.m. two-tailed Student's t-test.

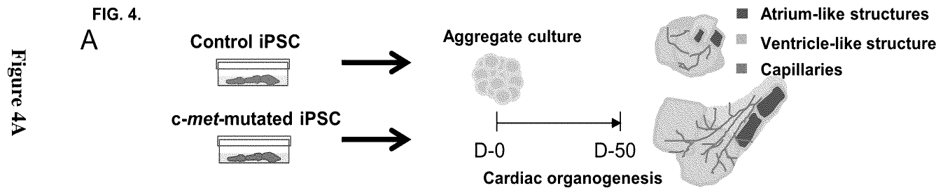

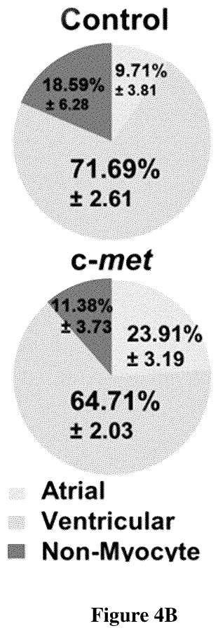

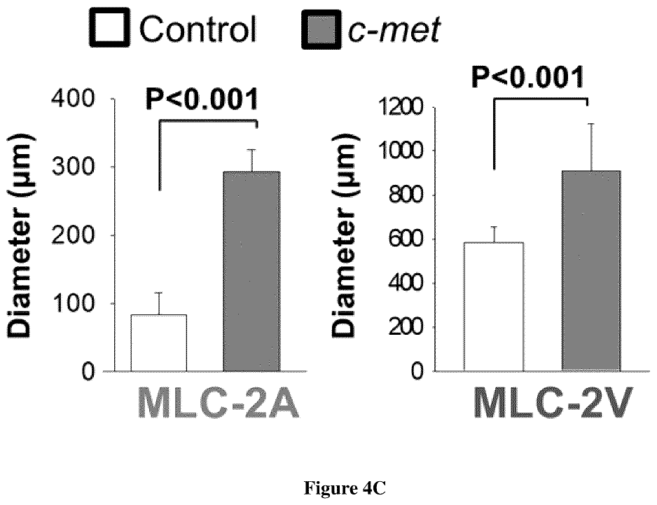

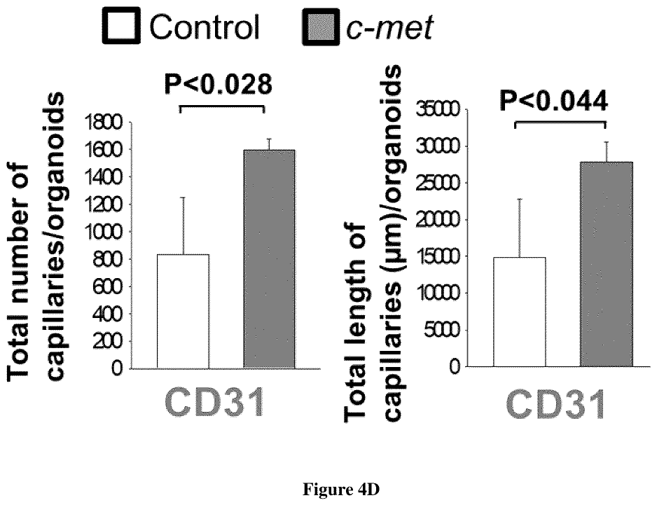

### FIG. 4

FIG. 4A: Schematic representation of the generation of vascularized cardiac chambers via matrix-free aggregation-mediated conditions.

FIG. 4B: The graph shows the mean percentage of DAPI+/MLC-2V+ (ventricular cardiomyocytes), DAPI+/MLC-2A+ (atrial cardiomyocytes) colocalization and only DAPI+ (non-myocytes) at 30-50 days.

FIG. 4C: The graph shows the mean diameter of MLC-2V+ cells area (control; n=30, c-met; n=27) and MLC-2A+ cells area (control; n=85, c-met; n=27).

FIG. 4D: The graph shows the total number and length of capillaries quantified, data normalized by organoids (n=3) at 30-50 days. Data are mean±s.e.m. two-tailed Student's t-test.

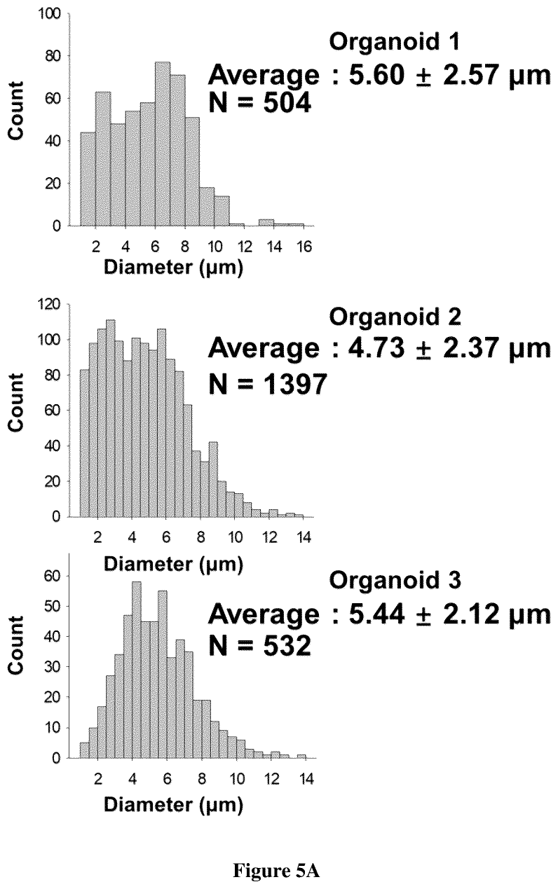

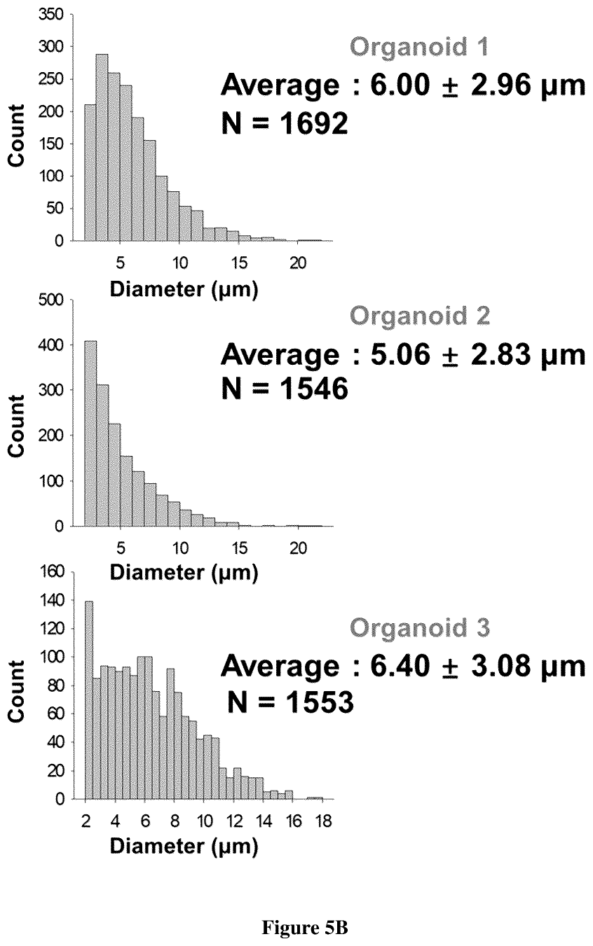

### FIG. 5

FIGS. 5A, 5B: Histograms of diameter of microvessels in control (FIG. 5A) and c-met (FIG. 5B) cardiac organoids. Scale bar: 100 μm.

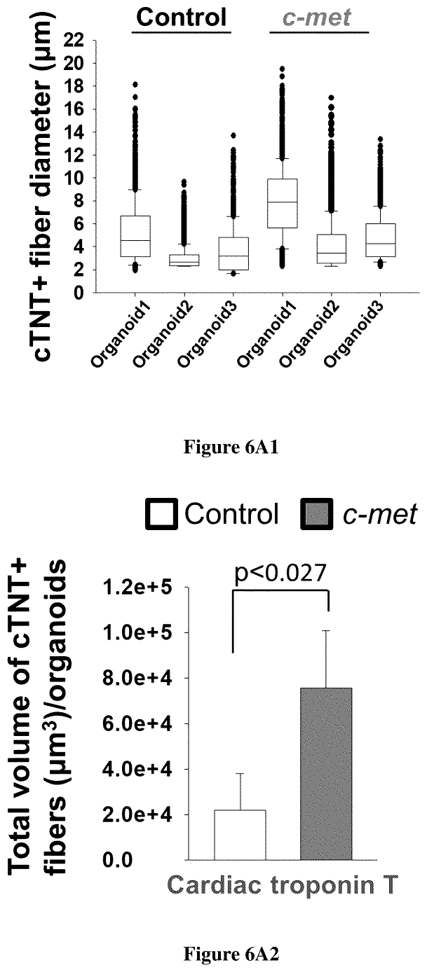

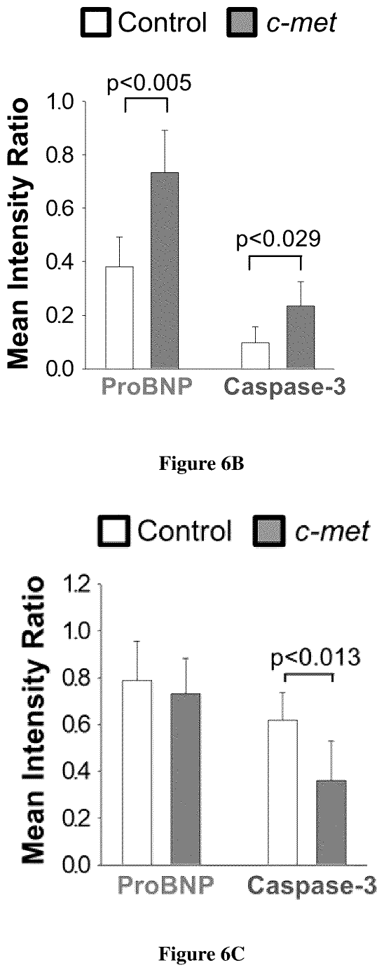

### FIG. 6

FIG. 6A1: Boxplot shows the diameter distribution of cTNT+fiber (FIG. 6A1) and the bar graph shows the total volume of cTNT+fiber (FIG. 6A2) (n=3) quantified, data normalized by organoids (n=3) at 30-50 days.

In control: organoid 1: median; 4.53 μm; min 1.94 μm; max 18.12 μm, organoid 2: median; 2.63 μm; min 2.27 μm; max 9.65 μm, organoid 3: median; 3.16 μm; min 1.63 μm; max 13.66 μm.

In c-met: organoid 1: median; 7.86 μm; min 2.27 μm; max 19.48 μm, organoid 2: median; 3.42 μm; min 2.27 μm; max 16.99 μm, organoid 3: median; 4.28 μm; min 2.27 μm; max 13.35 μm

FIG. 6 B: The graph shows the mean ProBNP and caspase-3 fluorescence intensity quantified, Data normalized by DAPI fluorescence intensity at 90-120 days. (FIG. 6B) normoxia. control: n=4, c-met: n=4.

FIG. 6C: cobalt chloride (200 μmol) induced hypoxia treatment for 24 h? control: n=5, c-met: n=8. All data are mean±s.e.m. two-tailed Student's t-test.

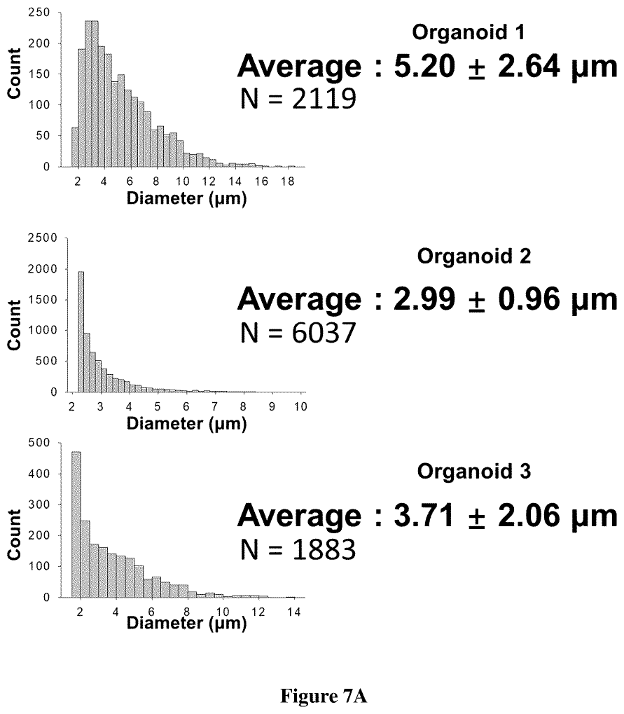

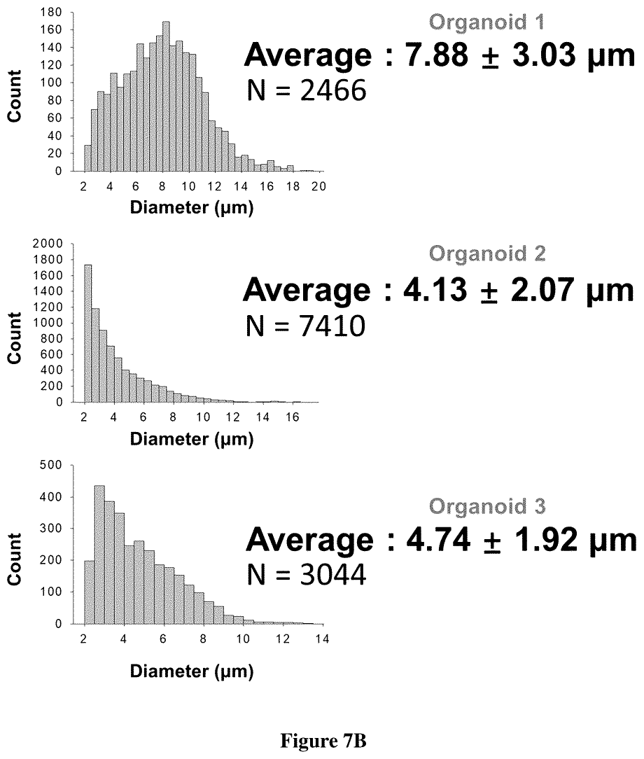

### FIG. 7

FIGS. 7A, 7B: Histograms of diameter of muscle fibers in control (FIG. 7A) and c-met (FIG. 7B) cardiac organoids. Scale bar: 100 μm.

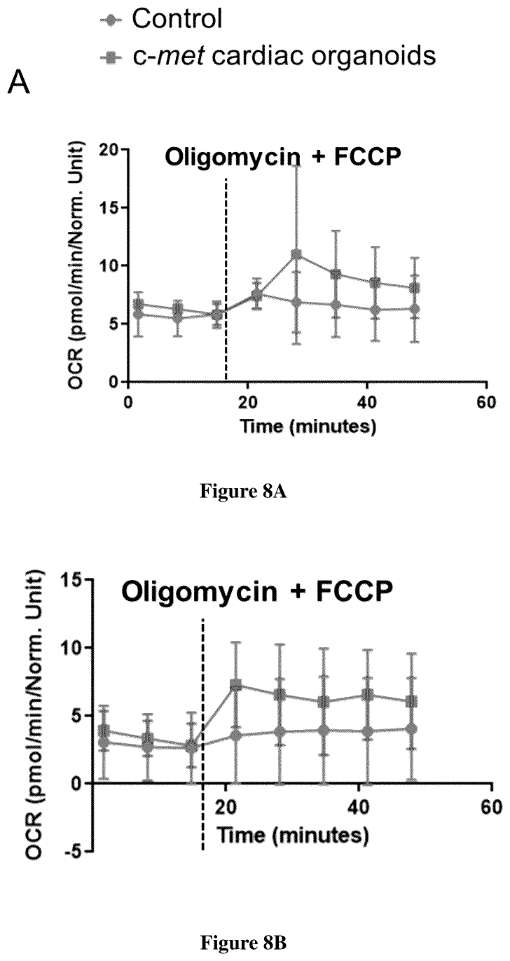

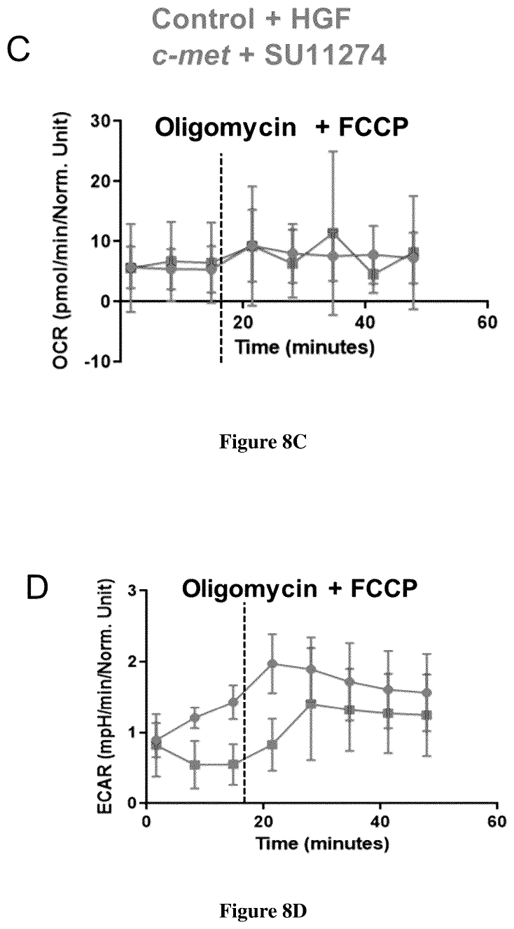

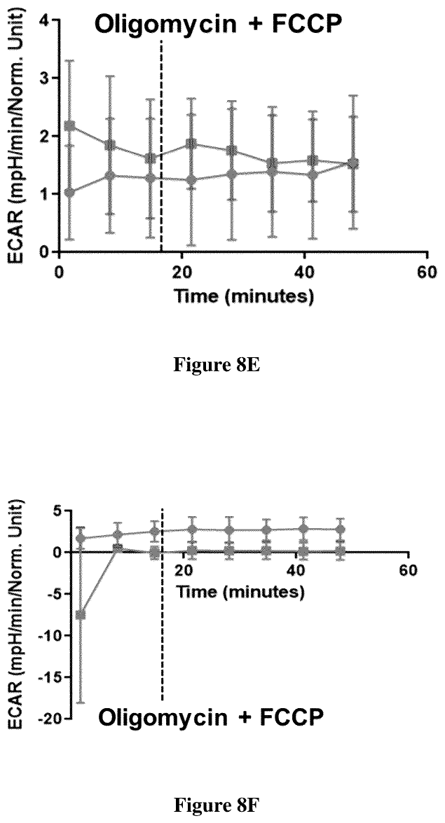

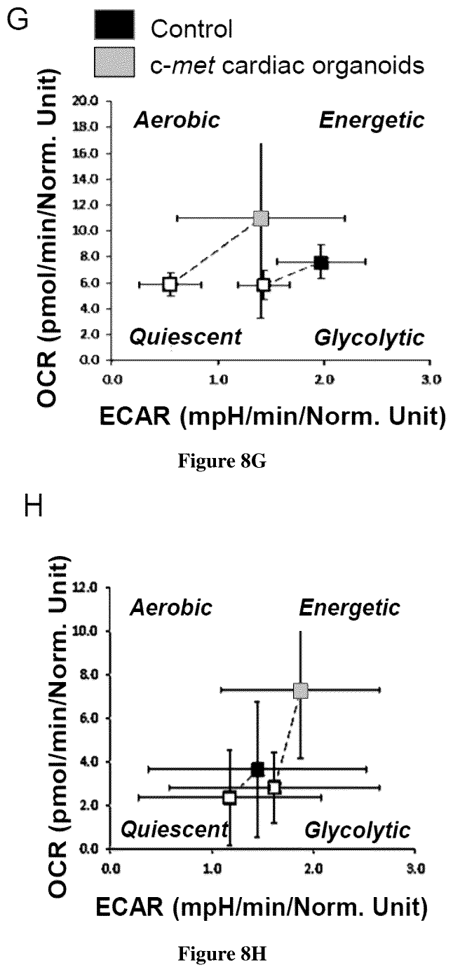

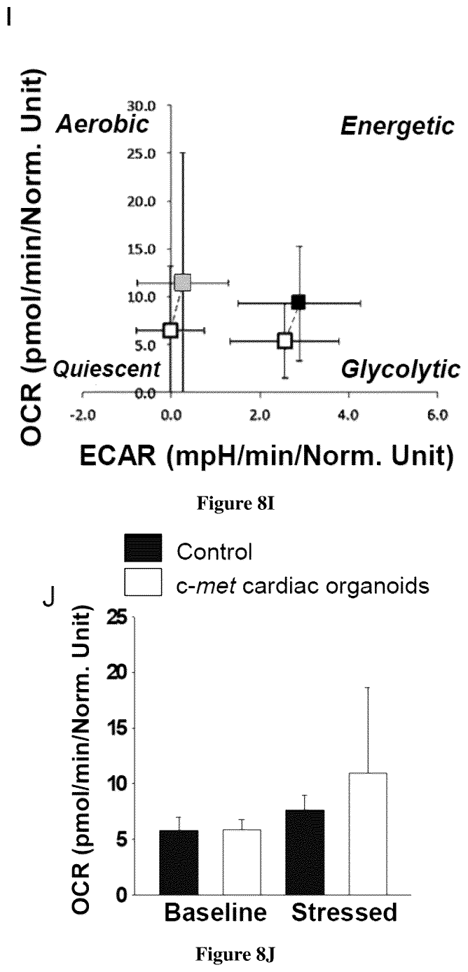

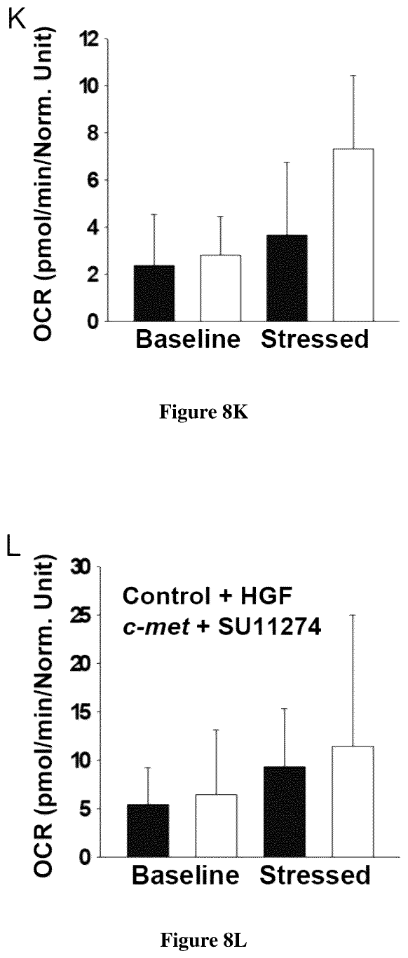

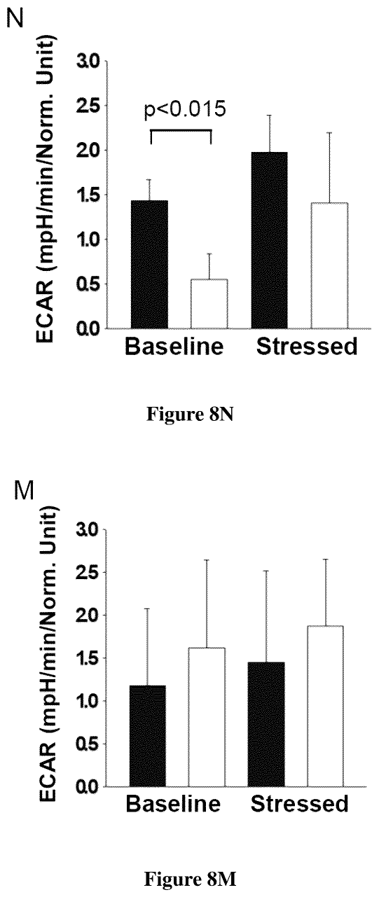

### FIG. 8

FIG. 8A, 8B, 8C, 8D, 8E, 8F: The real-time measurement of OCR and ECAR with injections of stressor compounds (n=3, mean±SEM). Control cardiac organoids: filled circles. c-met cardiac organoids: filled squares.

FIGS. 8G, 8H, 8I: Cell energy phenotype; OCR versus ECAR of control and c-met cardiac organoids grown in 96-well plate for 90-120 days.

FIG. 8G: left curve: c-met cardiac organoids. Right curve: control cardiac organoids

FIG. 8H: left curve: control cardiac organoids. Right curve: c-met cardiac organoids

FIG. 8I: left curve: c-met cardiac organoids. Right curve: control cardiac organoids

FIGS. 8J, 8K, 8L, 8N, 8M, 8O: The graph shows percentage of mean OCR and ECAR were calculated according to the manufacturer's protocol.

FIGS. 8B, 8E, 8H, 8K, 8M: Organoids were measured after treatment with CoCl2 (200 μmol) induced hypoxia for 24 hours.

FIGS. 8C, 8F, 8I, 8L, 8O: Control organoids were measured after treatment with CoCl2+HGF for 24 h and c-met cardiac organoids were measured after treatment with CoCl2+SU11274 for 24 h.

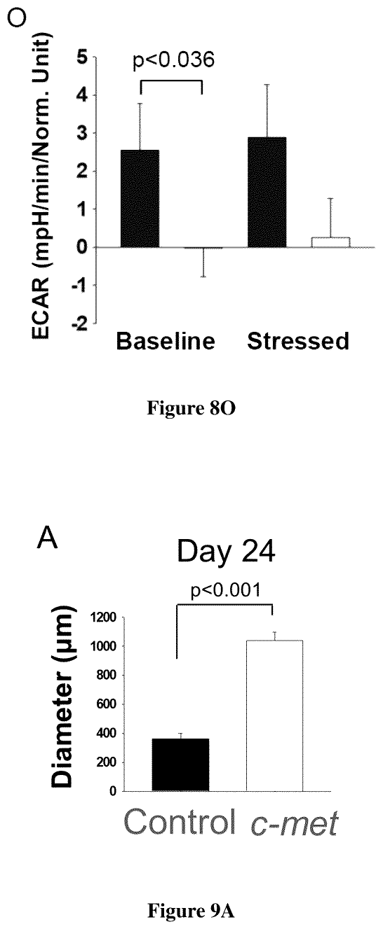

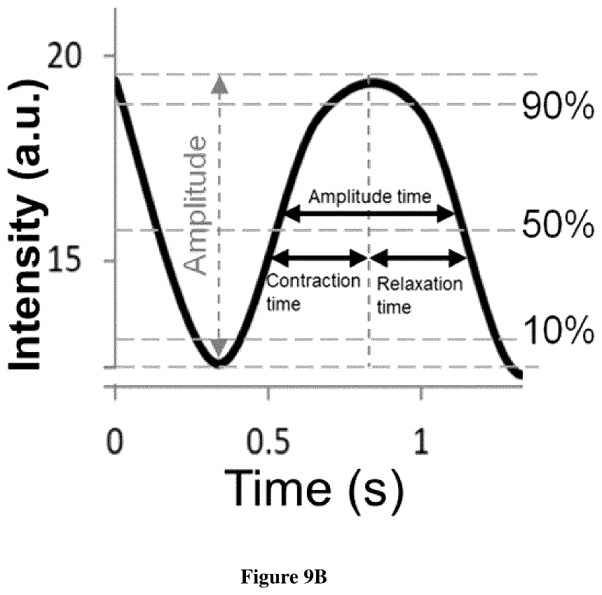

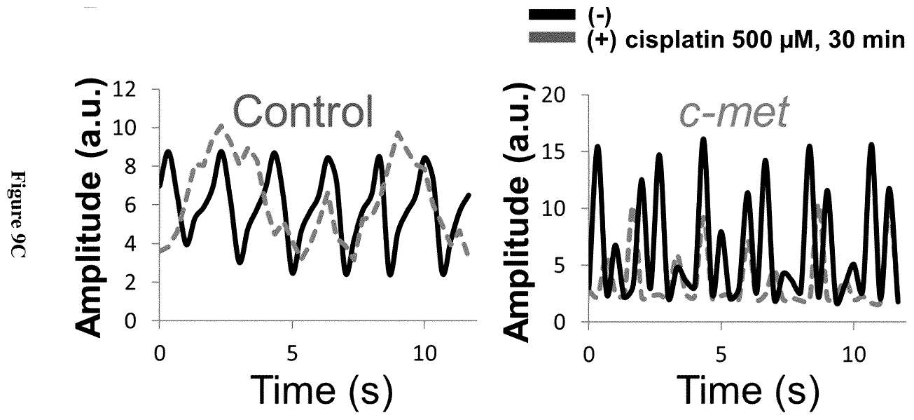

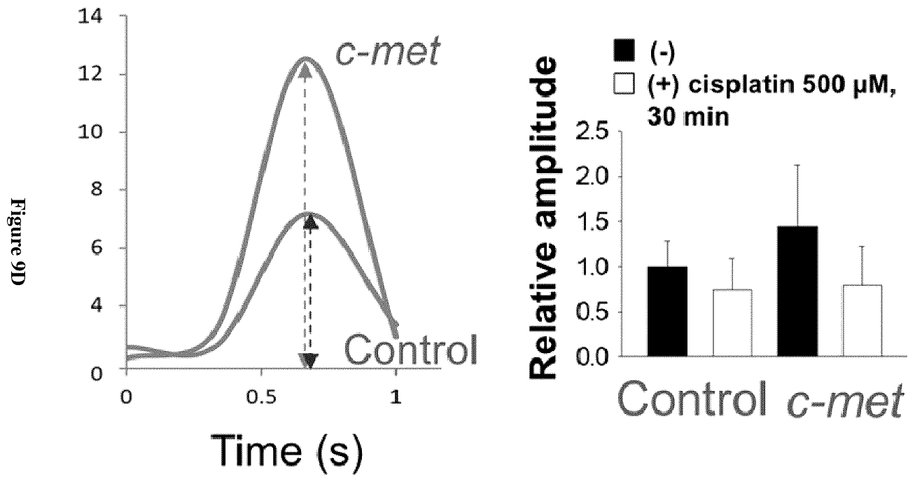

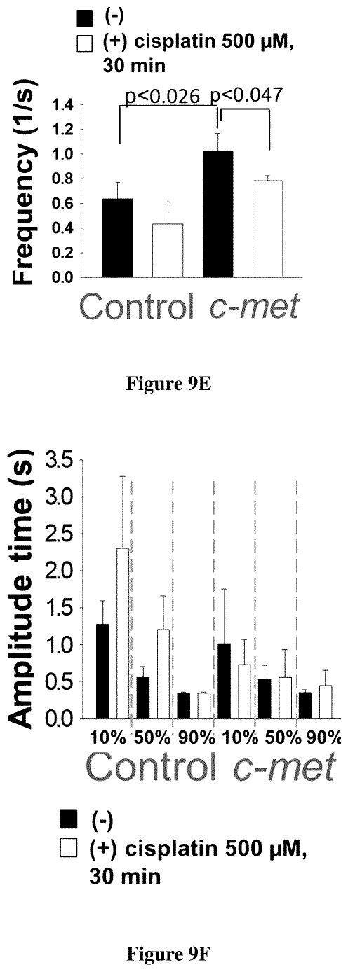

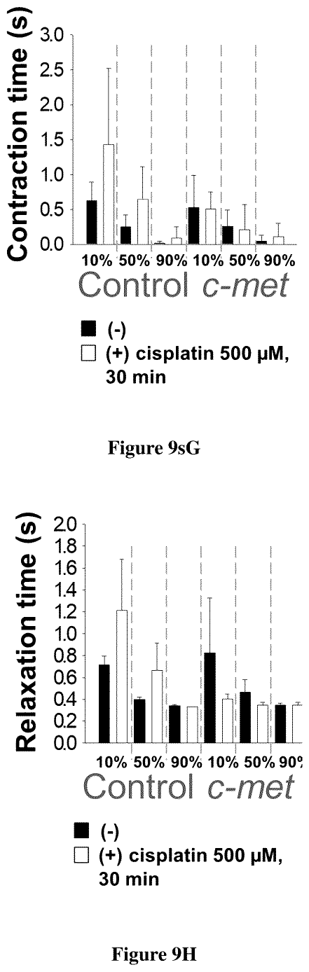

### FIG. 9

FIG. 9A: The graph shows diameter of randomly selected cardiac organoids (n=3) at 24 days for contractility assay.

FIG. 9B: Schematic representation of contraction parameters analyzed in organoids using myocyter and ImageJ software.

FIG. 9C: Amplitude of cardiac organoids in normal condition (black line) and after cisplatin (500 μmol/well) treatment for 30 min (dotted line).

FIG. 9D: The graph shows relative amplitude of contraction.

FIG. 9E: The graph shows frequency of contraction

FIGS. 9F, 9G, 9H: The graph shows amplitude time (FIG. 9F), contraction time (FIG. 9G), relaxation time (FIG. 9H) 10%, 50% and 90% (n=3/group.

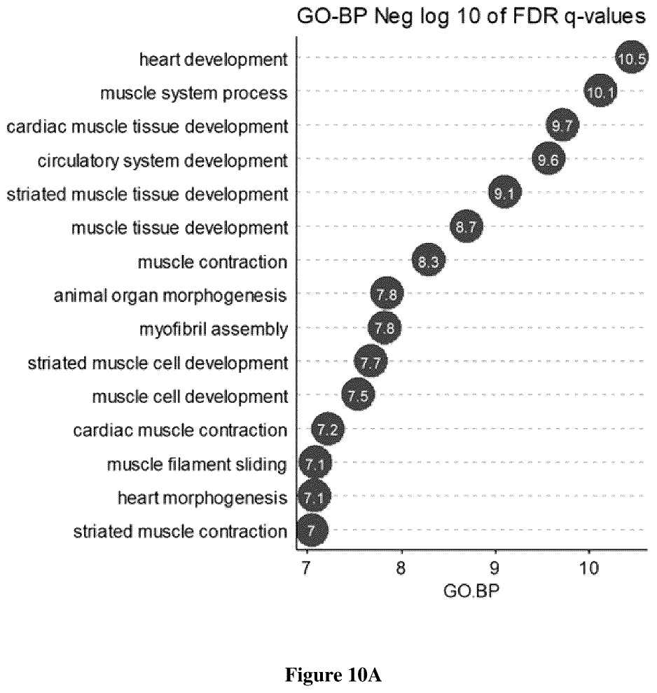

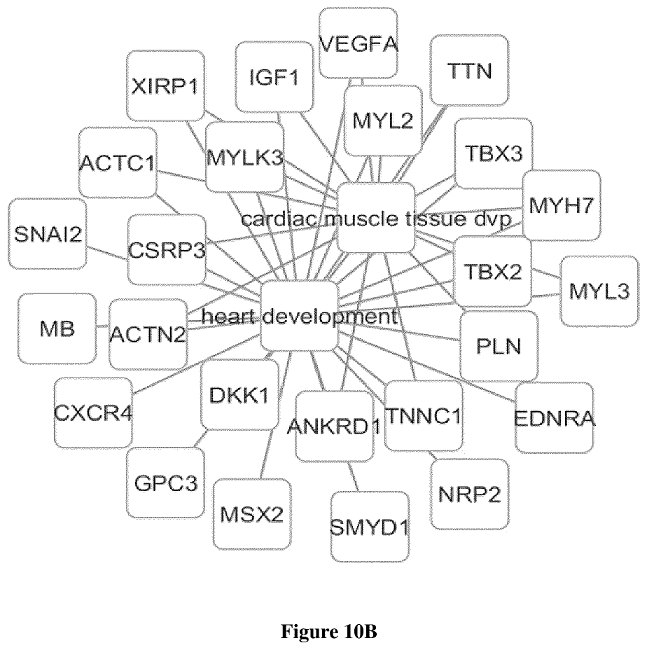

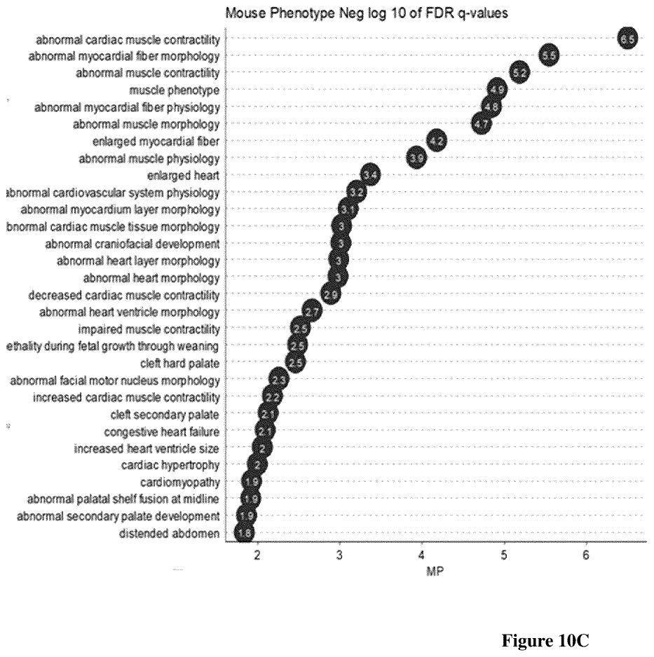

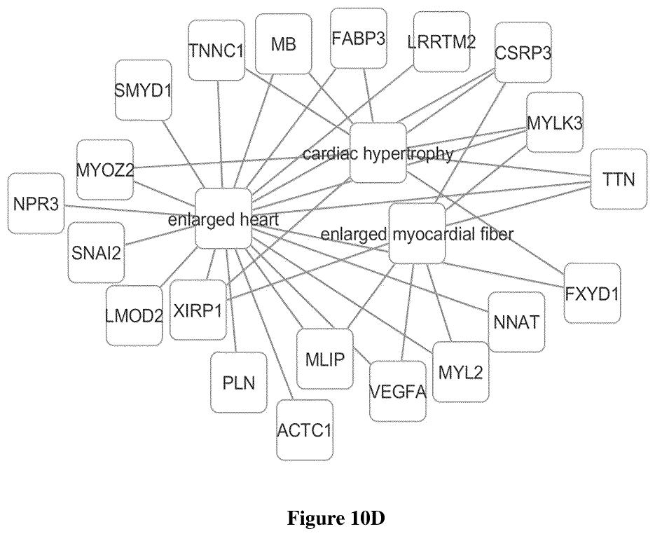

### FIG. 10

FIG. 10A: Functional enrichment on Gene ontology biological process database for genes up regulated in c-met cardiac organoids (results in negative log p-values of False Discovery Rate q-values).

FIG. 10B: Cardiac developmental functional enriched network up regulated in c-met cardiac organoids.

FIG. 10C: Functional enrichment on Mouse phenotype database for genes up regulated in c-met cardiac organoids (results in negative log p-values of False Discovery Rate q-values) . . . .

FIG. 10D: Cardiac hypertrophy functional enriched network up regulated in c-met cardiac organoids.

Abbreviations: VEGFA: Vascular Endothelial Growth Factor A; TTN: Titin; MYL2: Myosin Light Chain 2; MYL3: Myosin Light Chain 3; MYH7: Myosin heavy chain 7; TBX2: T-Box Transcription Factor 2; TBX3: T-Box Transcription Factor 3; PLN: Phospholamban; EDNRA: Endothelin Receptor Type A; TNNC1: Troponin C1; NRP2: Neuropilin 2; ANKRD1: Ankyrin Repeat Domain 1; SMYD1: SET And MYND Domain Containing 1; MSX2: Msh Homeobox 2; DKK1: Dickkopf WNT Signaling Pathway Inhibitor 1; GPC3: Glypican 3; CXCR4: C-X-C Motif Chemokine Receptor 4; ACTN2: Actinin alpha 2; MB: Myoglobin; CSRP3: Cysteine And Glycine Rich Protein 3; SNAI2: Snail Family Transcriptional Repressor 2; ACTC1: Actin; XIRP1: Xin Actin Binding Repeat Containing 1; MYLK3: Myosin Light Chain Kinase 3; IGF1: Insulin Like Growth Factor 1; FABP3: Fatty Acid Binding Protein 3; LRRTM2: Leucine Rich Repeat Transmembrane Neuronal 2; FXYD1: FXYD Domain Containing Ion Transport Regulator 1; NNAT: Neuronatin; MLIP: Muscular LMNA Interacting Protein; LMOD2: Leiomodin 2; NPR3: Natriuretic Peptide Receptor 3; MYOZ2: Myozenin 2.

### DETAILED DESCRIPTION OF THE INVENTION

The inventors have conceived and developed of a novel matrix-free and feeder-free method of generating mammal iPSC-derived cardiac organoids, which method includes both steps of dynamic culture conditions and static culture conditions.

As it is shown in the examples herein, cardiac organoids obtained with the matrix-free and feeder-free method disclosed herein are relevant models for testing potentially active substances on cardiac organoids obtained from iPSCs derived from subjects affected with a cardiac disease or disorder. The present disclosure relates to a matrix-free and feeder-free method for producing a cardiac organoid comprising the steps of:
a) providing mammal IPSCs,
b) culturing the mammal IPSCs provided at step a) under dynamic culture conditions during a time period ranging from 30 minutes to 3 hours, whereby formation of IPSCs cell aggregates is initiated,
c) culturing under static culture conditions the cell aggregates under formation obtained at step b), during a time period ranging from 12 hours to 48 hours, whereby IPSCs cell aggregates are obtained,
d) incubating the cell aggregates obtained at step c) in a cardiomyocyte differentiation medium, whereby a differentiated myocyte-containing organoid is obtained, and
e) culturing the differentiated myocyte-containing organoid obtained at step d), whereby a cardiac organoid is obtained.

The cardiac organoid obtained by the above method may be named a bioengineered mammal induced pluripotent stem cells-derived (IPSCs-derived) cardiac organoid.

As it is shown in the examples herein, the matrix-free and feeder-free method disclosed herein allows rapid generation of beating cardiac organoids in a short period of time, such as in less than 10 days. Especially, the inventors have shown that step b) of culturing the mammal iPSCs provided at step a) under dynamic culture conditions for the specified time period allow generating cardiomyocyte aggregates more rapidly and with a higher rate as compared to the same method where step d) is omitted.

Further, as it is also shown in the examples herein, long-term three-dimensional culture of cardiac organoids obtained by the method disclosed herein can give rise to cardiac organoids harboring blood vessels as well as atrium- and ventricular-like structures.

In the context of cardiac pathology, it is shown in the examples that the matrix-free method disclosed herein can be used to generate pathological cardiac organoids, such as cardiac organoids obtained from c-met-mutated iPSC. As it is well known in the art, c-met is proto-oncogene and hepatocyte growth factor receptor. Abnormal c-met signaling pathway is known to play an important role in the development of cardiac hypertrophy in mice models. The results contained in the examples show that the cardiac organoids obtained by the method disclosed herein may be used for testing existing or candidate agents for preventing or treating cardiac disorders or diseases.

### Definitions

Terms and phrases used in this application, and variations thereof, especially in the appended claims, unless otherwise expressly stated, should be construed as open ended as opposed to limiting.

It is to be noted that the term “a” or “an” entity refers to one or more of that entity; for example, “a iPSC cell,” is understood to represent one or more iPSCs cells. As such, the terms “a” (or “an”), “one or more,” and “at least one” can be used interchangeably herein.

The terms “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. The words “have” and “comprise,” or variations such as “has,” “having,” “comprises,” or “comprising,” will be understood to imply the inclusion of the stated element(s) (such as a composition of matter or a method step) but not the exclusion of any other elements.

The term “consisting of” implies the inclusion of the stated element(s), to the exclusion of any additional elements. The term “consisting essentially of” implies the inclusion of the stated elements, and possibly other element(s) where the other element(s) do not materially affect the basic characteristic(s) of the disclosure. It is understood that the different embodiments of the disclosure using the term “comprising” or equivalent cover the embodiments where this term is replaced with “comprising only”, “consisting of” or “consisting essentially of”.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “at least” refers to a situation in which a particular value is equal to or greater than that particular value. For example, “at least 2” is understood to be the same as “2 or more”.

As used herein, the terms “less than”, “less than or equal to”, etc. refer to the range from 0 to that value, including the values shown. For example, “less than 10” or “less than or equal to 10” encompasses values such as 0, 1, 2, 3, 4, 5, 6, 7, 8, 9 or 10.

Within the disclosure, the terms “significantly” or “substantially” used to qualify a difference or a change, for example ‘significantly different of” or “substantially different from”, with respect to a feature or a parameter intends to mean that the observe change or difference is noticeable and/or it has a statistic meaning. Conversely, the terms significantly” or “substantially” used to qualify a similitude or an identity, for example “not significantly different from” or “substantially identical to”, with respect to a feature or a parameter intends to mean that any observed change or difference is such that the nature and function of the concerned parameter or feature is not materially affected.

As used herein, the term “mammal” encompasses both a non-human mammal and a human subject. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In most preferred embodiments a “mammal” is a human. A mammal can be any age or at any stage of development (e.g., an adult, teen, child, infant). A mammal can be male or female.

The term “bioengineered” is used herein to describe a man-made organ or tissue engineered to have biological properties similar or identical to a naturally occurring organ or tissue. In some aspects, this may require the use of engineering of a particular apparatus; in other aspects, this may require the use of a variety of biological factors.

As used herein, the term “stem cell” is a cell having the ability to self-regenerate and differentiate at the single cell level to produce progeny cells, including self-regenerating progenitor cells, non-regenerating progenitor cells and terminally differentiated cells. This term refers to undifferentiated cells that have the ability to divide indefinitely in culture. Stem cells are also characterized by their ability to contribute substantially, if not all, to most tissues. Stem cells are classified as somatic (adult) stem cells or embryonic stem cells. Somatic stem cells are undifferentiated cells found in differentiated tissues that can regenerate (clone) themselves and differentiate (to some extent) to give rise to all specialized cell types of tissues from which they originate. be.

As used herein, the term “pluripotency” is commonly understood by those of skilled in the art as the capacity of a stem cell to differentiate into one or more tissues or organs, including heart tissue.

The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a mammal). Pluripotent cells are capable of forming teratomas and of contributing to ectoderm, mesoderm, or endoderm tissues in a living organism.

By “induced pluripotent stem cell” or “iPSC” it is meant a PSC that is derived from a cell that is not a PSC (i.e., from a cell that is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors known in the art to reprogram the somatic cells to become pluripotent stem cells.

As used herein, “iPS cell derivation” means reprogramming a somatic cell to become pluripotent.

As used herein, the term “differentiation” refers to non-specialized iPSCs differentiating in specialized cells such as heart cells (e.g. cardiomyocytes), under control conditions in in vitro culture. This term refers to the biological process of acquiring adult cell characteristics.

As used herein, the term “cardiomyocyte” usually refers to cardiomyocyte lineage cells consisting of mature cardiomyocytes or adult-like cardiomyocytes. As intended herein cardiomyocytes encompass atrial cardiomyocytes and ventricular cardiomyocytes. Most preferably herein, the term “cardiomyocyte” refers to atrial cardiomyocyte and ventricular cardiomyocytes.

As used herein, the term “suspension culture” refers to a cell culture, individual cells or cell clusters, e.g. cell aggregates under formation, suspended in medium, and not attached to any surface.

As used herein, cells, especially iPSCs, are cultivated under “dynamic conditions” when these cells are cultivated under conditions in which they constantly move in suspension culture.

As used herein, cells, especially iPSCs, are cultivated in “static conditions” or “non-dynamic conditions” when these cells are cultivated under conditions in which they do not move in suspension culture.

The term “aggregates,” refers to cell clusters comprising differentiated and partly differentiated cells that appear when pluripotent stem cells are allowed to differentiate in a non-specific fashion.

The term “cardiomyocyte differentiation medium” means any medium that is suitable for causing differentiation of iPSCs, including iPSCs comprised in cell aggregates, into cardiomyocytes, without any restriction as to the mode of action.

The term “cardiomyocyte maintenance medium” means any medium that is suitable for maintenance of cardiomyocytes, without any restriction as to the mode of action.

As used herein, the term “organoid” refers to a small culture that reproduces both the form and function of a tissue or organ. More specifically, an organoid must contain one or more cell types among various types of cells constituting an organ or tissue, and the cells must be spatially agglomerated with each other. Organoids can be used as a patient-specific model for drug development in view of conceiving disease treatments.

As used herein, the term “cardiac organoid” refers to an organoid comprising cardiomyocytes, and possibly also epithelial/vascular cells.

In the present invention, the term “marker” as used herein refers to a property that is specifically associated with the phenotype of the cell. It can be used to assess differentiation into sequences. For example, a “marker” can refer to a nucleic acid or polypeptide molecule that is discriminatoryly expressed in the cell. The detectable level of the marker nucleic acid or polypeptide is sufficiently higher in the cell as compared to other cells so that the cell can be identified using various methods known in the art and distinguished from other cells. Is low.

As used herein, the term “serum-free medium” is commonly understood by those of skill in the art and refers to a medium that is substantially serum-free. By definition, serum-free medium lacks whole serum as a component, but serum-derived products may not be completely excluded. For example, high-purity albumin, such as bovine or human (recombinant) albumin, may be included in the serum-free medium. For example, it may contain up to 10% by weight, preferably up to 5% by weight, even more preferably up to 2% by weight, up to 1% by weight, up to 0.5% by weight, most preferably up to 0.25% by weight of albumin.

As used herein, the term “matrix-free”, means that a referred method does not comprise any step wherein cells are cultured in the presence of a support matrix or scaffold.

As used herein, the term “feeder-free” means that a referred method does not comprise any step wherein cells are cultured in the presence of feeder cells, e.g. any step wherein cells are cultured in the presence of a layer of feeder cells.

As used herein, the term “subject” refers to any mammal, but typically mammals such as human mammals, pets (eg dogs or cats), domestic animals (eg horses, cows). Or sheep), or experimental animals (eg, rats, mice, non-human primates or guinea pigs). In a preferred embodiment, the subject is a human, preferably an adult.

### DETAILED DESCRIPTION

The present disclosure relates to an in vitro matrix-free method for producing a mammal cardiac organoid comprising the steps of:
a) providing mammal iPSCs,
b) culturing the mammal iPSCs provided at step a) under dynamic culture conditions during a time period ranging from 30 minutes to 3 hours, whereby formation of iPSCs cell aggregates is initiated,
c) culturing under static culture conditions the cell aggregates under formation obtained at step b), during a time period ranging from 12 hours to 48 hours, whereby iPSCs cell aggregates are obtained,
d) incubating the cell aggregates obtained at step c) in a cardiomyocyte differentiation medium, whereby a bioengineered mammal iPSCs-derived cardiac organoid is obtained, and
e) culturing the differentiated myocyte-containing organoid obtained at step d), whereby a cardiac organoid is obtained.

It is specified that steps a) to e) of the method shall imperatively be performed successively, according to the indicated order.

The cardiac organoid that is obtained at step e) can thus also be termed a mammal bioengineered iPSCs-derived cardiac organoid, herein.

### Step a) of the Method

Step a) of the method comprises, or consists of, providing mammal, most preferably human, induced pluripotent stem cells (also termed “iPSCs” or “iPSCs cells” herein).

Numerous lines of iPSCs are available to the skilled person.

Further, numerous methods for obtaining iPSCs are known in the art, especially methods for programming (i.e. “reprogramming”) differentiated cells obtained from a subject.

Thus, at step a) of the method, it can be used inducible pluripotent cells (iPSCs), i.e. reprogrammed pluripotent cells, that can be obtained from reprogramming adult somatic cells.

As it is well known in the art, induced pluripotent stem (iPSCs) cells are cells which have the characteristics of ES cells but are obtained by the reprogramming of differentiated somatic cells. Induced pluripotent stem cells have been obtained in the art by various methods.

The induced pluripotent stem cells are morphologically similar to human ES cells, and express various human ES cell markers. Also, when grown under conditions that are known to result in differentiation of human ES cells, the induced pluripotent stem cells differentiate accordingly. For example, the induced pluripotent stem cells can differentiate into cells having cardiomyocyte structures and cardiomyocyte markers. It is anticipated that virtually any iPSCs cells or cell lines may be used at step a) of the disclosed method.

Methods of preparing induced pluripotent stem cells from mouse are also known (e.g. Takahashi and Yamanaka, 2006, Cell, Vol. 126 (4):663-676). Induction of iPSCs cells typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc.

Reprogramming factors may be expressed from expression cassettes comprised in one or more vectors, such as an integrating vector or an episomal vector, such as a EBV element-based system (see PCT application WO 2009/149233). In a further aspect, reprogramming proteins could be introduced directly into somatic cells by protein transduction.

A number of mammal iPSCs cell lines, including a number of human iPSCs cell lines, are available from a plurality cell line collections throughout the world. Mammal iPSCs lines, including human iPSCs lines are available at the American Type Culture Collection (ATTC, VA, USA).

For performing step a) of the disclosed method, the skilled artisan may refer to the iPSCs lines described by Hwang et al., 2019, Int. J. Mol. Sci., Vol. 20, doi:10.3390/ijms20194867).

Thus, in some embodiments, the mammal iPSCs provided at step a) derive from the programmation of a sample of differentiated human cells.

As it will be described later in the present disclosure, the mammal iPSCs provided at step a) derive from the programmation of a sample of differentiated human cells originating from a subject affected with a cardiac disease.

For providing mammal iPSCs, especially human iPSCs, at step a) of the method, the said iPSCs can be previously cultured in an appropriate culture medium, either in the presence of feeder cells, or alternatively in feeder-free conditions. Both kinds of culture conditions are largely documented in the art. The skilled artisan may refer to the review article by Yu et al. (2014, in: Interface Oral Health Science, Keiichi Sasaki, Osamu Suzuki, Nobuhiro Takakashi eds, Springer Verlag, pp. 145-159). Beyond culture conditions, these authors disclose a plurality of available feeder cells sources.

For culturing the iPSCs in culture conditions involving feeder cells, the skilled artisan may refer to the article of Yue et al. (2012, PLOSONE, Vol. 7:e32707).

In some embodiments, the iPSCs provided at step a) can have been previously cultured in feeder-free conditions, such as on a matrix basal membrane, for example on a growth factor-containing basement membrane, such as the matrix basal membranes commercially available as Matrigel™ of Geltrex™.

As it is known in the art, various matrix components may be used in culturing and maintaining human pluripotent stem cells. For example, collagen IV, fibronectin, laminin, and vitronectin in combination may be used to coat a culturing surface as a means of providing a solid support for pluripotent cell growth, as described in Ludwig et al. (2006a; 2006b), which are incorporated by reference in their entirety.

Matrigel™ may also be used to provide a substrate for cell culture and maintenance of human pluripotent stem cells. Matrigel™ is a gelatinous protein mixture secreted by mouse tumor cells and is commercially available from BD Biosciences (New Jersey, USA). This mixture resembles the complex extracellular environment found in many tissues and is used by cell biologists as a substrate for cell culture.

In preferred embodiments, the iPSCs that are provided at step a), when previously cultured according to feeder-free conditions, can be cultured on the basement membrane Geltrex™, as it is illustrated in the examples herein.

In preferred embodiments, the iPSCs that are provided at step a) were previously cultured in a, “E8” medium that is well known in the art.

The terms “E8 medium”, or simply “E8” are used interchangeably herein to refer to a specific medium formulation for the culture of iPSCs, at step a) of the disclosed method. An E8 medium comprises, in addition to the components of the commercially available solution of DMEM/F12 with L-Glutamine and HEPES, a final concentration of about 543 μg/ml sodium bicarbonate, about 64 μg/ml L-ascorbic acid 2-phosphate, about 14 ng/ml sodium selenite, about 10.7 μg/ml recombinant human transferrin, about 20 μg/ml recombinant human Insulin, about 100 ng/ml recombinant human FGF2, and about 2 ng/ml recombinant human TGFB1.

For the sake of clarity, the fact that, in some embodiments, the iPSCs provided at step a) of the disclosed method were previously cultured (i) on a feeder cell layer or (ii) on a matrix basement membrane does in no case means that the disclosed method does not consist of a matrix-free and feeder-free method. Indeed, the skilled artisan readily understands that step a) consists of providing previously cultured iPSCs, irrespective of the culture conditions that have allowed availability of the iPSCs that are subsequently provided at step a) of the method.

At step a), the mammal iPSCs, most preferably consist of human iPSCs.

At step a), the mammal iPSCs, most preferably the human iPSCs, are preferably provided as a collection of single cells, that can be seeded at the desired cell density, at step b) of the disclosed method.

Single-cell dissociation of pluripotent stem cells followed by single cell passaging may be used with several advantages, like facilitating cell expansion,

In certain embodiments, cultured pluripotent stem cells may be dissociated into single individual cells, or a combination of single individual cells. The dissociation can be achieved by mechanical force, or by a cell dissociation agent, such as NaCitrate, or an enzyme, for example, trypsin, trypsin-EDTA, TrypLE Select, or the like, according to techniques well known in the art.

In preferred embodiments, the iPSCs provided at step a) were previously obtained by reprogramming human bone marrow mononuclear cells from a subject, such as described by Hwang et al. (2019, Int. J. Molecular Sciences, Vol. 20:4867).

Once provided at step a), the iPSCs are processed according to the further steps of the disclosed method.

### Step b) of the Method

At step b) of the disclosed method, the mammal iPSCs provided at step a) are cultured under dynamic culture conditions, so as to initiate formation of iPSCs aggregates.

At step b), the iPSCs provided at step a) are cultivated under conditions in which they constantly move in suspension culture.

Importantly, the whole steps of the disclosed method, including step b), are performed by using a unique source of cells; the iPSCs that are provided at step a) of the method, thus by method steps of cell “monoculture”, without any step of co-culturing with any other cell.

Otherwise said, at no step of the disclosed method is this unique source of cell co-cultured with one or more other cells, such as for example fibroblasts or endothelial cells.

In preferred embodiments, the iPSCs are seeded at the beginning of step b) ate a cell density ranging from 10⁴ cells/mL to 10⁷ cells/mL

As used herein, a cell density ranging from 10⁴ cells/mL to 10⁷ cells/mL encompasses cell density values of 10⁴ cells/mL, 0.5 10⁵ cells/mL, 10⁵ cells/mL, 0.5 10⁶ cells/mL, 10⁶ cells/mL, 0.5 10⁷ cells/mL and 10⁷ cells/mL

Without wishing to be bound by any particular theory, the inventors believe that seeding the iPSCs at a cell density lower than 10⁴ cells/mL, at the beginning of step b), does not allow aggregates to be easily initiated, due to a low likelihood of the seeded cells to be contact.

Without wishing to be bound by any particular theory, the inventors believe that seeding the iPSCs at a cell density higher than 10⁷ cells/mL, at the beginning of step b), may lead to initiate the formation of somewhat unorganized aggregates that may subsequently lead to organoids devoid of the atrial-like and ventricular-like chambers which are sought.

Accordingly, at step b), iPSCs are kept in motion in order to initiate the formation of iPSCs cell aggregates.

For performing step b), useful dynamic suspension culture systems include any systems known in the art equipped with means for maintaining movement of the cultured iPSCs, for example, by mixing, shaking, recirculating, or passing gases through the culture medium in which the iPSCs are being cultured.

The dynamic culture conditions can be implemented according to any cell culture method wherein cells are kept in movement in the culture medium at a controlled moving speed, by using, for example, shaking, rotating, or stirring platforms or culture vessels.

The agitation may also improve circulation of nutrients and cell waste products and is used, at step b) of the disclosed method, to control iPSCs cell aggregation by providing a more uniform environment. Using dynamic culture conditions at step b) of the disclosed method allows initiation of self-organization and self-pattering of the aggregates with acceleration of organoid production.

In preferred embodiments, dynamic culture conditions are performed by orbital shaking of the culture containers, e.g. in culture plates known in the art such as non adherent 12-well, 24-well or 96-well culture plates.

At low strength dynamic culture conditions, such as at low rotary speed, the rate at which iPSCs initiate aggregation is low, which induces that step b) is preferably performed during a long period of time.

At high strength dynamic culture conditions, such as et high rotary speed, the rate at which iPSCs initiate aggregation is higher than at low strength, which induces that step b) is preferably performed during a short period of time.

Rotary speed is preferably ranging from 10 to 100 rounds per minute (“rpm”). For example, rotary speed may be set to about 10, 15, 20 25, 30, 35, 40, 45, 50, 55, 60, 65, 70, 75, 80, 85, 90, 95 or 100 rpm, or any range derivable thereof.

Without wishing to be bound by any theory, the inventors believe that dynamic culture conditions performed by an orbital shaking at a rotary speed lower than 10 rpm will cause at least part of the cultured iPSCs to sediment, which sedimentation will significantly alter iPSCs aggregate formation.

Without wishing to be bound by any theory, the inventors believe that dynamic culture conditions performed by an orbital shaking at a rotary speed lower than 100 rpm will prevent iPSCs aggregate formation, due to an excessive shearing force preventing intercellular attachment and thus preventing an appropriate seedling of the aggregates, so as to suitably initiate the formation of iPSCs aggregates.

Preferably, the rotary speed ranges from 50 rpm to 90 rpm.

In some preferred embodiments, step b) can be performed during about 1 hour under orbital shaking at about 70 rpm.

Such cell culture agitation can be intermittent or continuous., although it is most preferably continuous.

Most preferably, the agitation speed, e.g. the rotary speed, is maintained at a constant value during the entirety of step b).

Step b) can be performed by cultivating the iPSCs in an appropriate dynamic culture system, such as disposable plastic, reusable plastic, made of stainless steel or glass vessels, such as a centrifuge tube or an Erlenmeyer flask. Step b) can be performed in in culture plates known in the art such as non adherent 12-well, 24-well or 96-well culture plates.

Thus, it shall be understood that step b) of the disclosed method comprises, or consists of, culturing only the mammal iPSCs provided at step a) under dynamic culture conditions during a time period ranging from 30 minutes to 3 hours, whereby formation of iPSCs aggregates is initiated.

In preferred embodiments, step b) is performed during a time period of about 1 hour.

Notably, the duration of step b) may depend on the strength of the dynamic culture conditions that are used, such as may depend from the rotary speed when orbital shaking is performed.

As it shall be easily understood, step b) is performed in matrix-free and feeder-free conditions, with the cultured cells of the cultured iPSCs aggregates initiating their formation, moving freely in the culture medium volume, due to the dynamic culture conditions.

The other culture conditions for step b) are those which are conventionally used in the art, notably for culturing iPSCs.

Notably, the iPSCs can be cultivated in any known appropriate culture medium, such as the commercially available “E8” medium well known in the art.

Generally, step b) is performed at a temperature appropriate for the cultured iPSCs, such as at about 37° C.

### Step c) of the Method

At step c), the iPSCs aggregates under formation obtained at step b) are cultured under static conditions during a time period preferably ranging from 12 hours to 48 hours, so as to generate iPSCs aggregates.

At step c) formation of the iPSCs aggregates may be easily observed, such as by using an inverted microscope device.

Step c) can be performed by cultivating the iPSCs in an appropriate static culture system, such as disposable plastic, reusable plastic, made of stainless steel or glass vessels, such as a centrifuge tube or an Erlenmeyer flask.

In preferred embodiments, step b) and step c) are performed in the same culture container, such as in the same 12-well, 24-well or 96-well low attachment culture plate, thus without cell transfer from a first culture container to a second culture container.

Step c) is performed during a period of time of about 12, 14, 16, 18, 20, 22, 24, 28, 30, 32, 34, 36, 38, 40, 42, 44, 46 or 48 hours.

It is believed that performing step c) for a period of time of less than 12 hours does not allow the formation of sufficiently complete iPSCs aggregates, which will prevent the subsequent formation of physiologically relevant cardiac organoids at the end of the disclosed method.

Further, it is believed that performing step c) for a time period higher than 48 hours does not bring any improvement in the size or completion of the iPSCs aggregates. In contrast, lengthening the duration of step c) beyond 48 hours may cause a decrease in iPSCs viability within the aggregates and thus will prevent formation of physiologically optimal cardiac organoids at the end of the disclosed method.

As it shall be easily understood, step c) is performed in matrix-free and feeder-free conditions, with the cultured iPSCs aggregates laid on the bottom surface of the culture container; e.g. on the bottom non adherent surface of 12-well, 24-well or 96-well culture plates.

The other culture conditions for step c) are those which are conventionally used in the art, notably for culturing iPSCs.

Notably, the iPSCs can be cultivated in any known appropriate culture medium, such as the commercially available “E8” medium well known in the art.

In some embodiments, the cardiac aggregates obtained at the end of step c) of the disclosed method, when obtained from IPSCs deriving from subjects devoid of cardiac hypertrophy, have a mean diameter ranging from 50 μm to 100 μm. The size of the aggregates may reach a mean diameter of 400 μm or more, when obtained from IPSCs deriving from subjects affected with a cardiac hypertrophy, such as from subjects bearing a mutated c-met gene.

### Step d) of the Method

Step d) of the disclosed method comprises, or consists of, incubating the cell aggregates obtained at step c) in a cardiomyocyte differentiation medium, whereby a differentiated myocyte-containing organoid is obtained.

At step d), any differentiation medium that is known in the art to be suitable for differentiating pluripotent stem cells into cardiomyocytes can be used.

Preferably, the cardiomyocyte differentiation medium comprises appropriate amounts of one or more differentiation factors for differentiating cardiomyocytes.

Also, using his background knowledge, the skilled artisan can determine the appropriate conditions of amount and/or timing of growth factors that should be added for any given differentiation medium batch. According to such an embodiment, determining the appropriate conditions comprises determining amounts of addition of one or more differentiation factors appropriate for differentiation of pluripotent cells into cardiomyocytes. This determination may comprise testing differentiation of iPSCs cells in a culture medium from the selected batch added with varied amounts of differentiation factors during a test period. For example, varied amounts of differentiation factors may be added during a test period.

For differentiating pluripotent cells, especially iPSCs, into cardiomyocytes, and without wishing to be bound by any theory, it is contemplated that TGFβ signaling pathways may be delicately regulated by adjusting the external addition of certain growth factors to achieve optimal cardiac differentiation condition. In particular, BMP signaling and Activin signaling are two exemplary TGFβ signaling pathways that can be optimized for the particular batch of culture medium employed. For example, BMP signaling inhibitor comprises dorsomorphin and Activin signaling inhibitor comprises SB431542. The cardiomyocyte differentiation medium may also comprise externally adjusted fibroblast growth factor (FGF), hepatocyte growth factor or any other differentiation factors that to be used or screened.

A plurality of differentiation media that are appropriate for differentiating pluripotent cells, including iPSCs, into cardiomyocytes are known in the art. It may notably referred to the media disclosed by Xu et al. (2008, Differentiation, Vol. 76:958-970), by Burridge et al. (2015, Current Protocols, doi.org/10.1002/0471142905.hg2103s87), by Lian et al. (2015, Nature Methods, Vol. 12:595-596), Balafkan et al. (2020, Nature, Scientific Reports, Vol. 10: 18498) or by Passier et al. (2005, Current Opinion in Biotechnology, Vol. 16 (5):498-502).

A plurality of commercial cardiomyocyte differentiation media may also be used at step c) of the disclosed method, such as (i) the differentiation kit marketed under the name STEMdiff™ by STEMCELL Technologies company or (ii) the PSC Cardiomyocyte Differentiation Kit marketed by ThermoFisher Scientific company).

Typically, the duration of step d) ranges from 1 to 5 days, such as 1,5 to 2.5 days, which includes about 2 days.

In preferred embodiments, step d) is performed by incubating the iPSCs aggregates obtained at step c) successively in more than one cardiomyocyte differentiation medium.

In preferred embodiments, step d) is performed by using the commercial PSC Cardiomyocyte Differentiation Kit marketed by ThermoFisher Scientific company), which comprises two differentiation media, (i) the Cardiomyocyte Differentiation Medium A and (ii) the Cardiomyocyte Differentiation Medium B, respectively (also termed “PSC-A” and “PSC-B” herein). For the qualitative and quantitative constitution of the differentiation media A and B, the skilled artisan may refer to the article by Blurridge et al. (2014, Nature Methods, Vol. 11:855-860).

In the embodiments of step d) wherein the cardiomyocyte differentiation medium consists of the combined PSC-A and PSC-B described above, step d) comprises, or consists of, incubating the iPSCs aggregates obtained at step c) with medium PSC-A and then medium PSC-B, successively. According to these embodiments, the iPSCs aggregates obtained at step c) are first incubated at step d) with medium PSC-A, during a time period ranging from 20 h to 30 h, most preferably a time period of about 24 h. Then, the medium PSC-A is removed and the differentiating cells are incubated with medium PSC-Bn during a time period of 20 h to 30 h, most preferably a time period of about 24 h.

Cardiac organoids comprising differentiated myocytes, also termed “differentiated myocyte-containing organoids” herein, are obtained at the end of step d)

### Step e) of the Method

Maturity of the cardiac organoids obtained at step d) of the disclosed method can be brought to completion by maintaining the differentiated myocyte-containing organoids obtained at step d) in appropriate culture conditions.

At step e), the differentiated myocyte-containing cardiac organoids obtained at step d) can be maintained in culture for a long period of time.

At step e), the differentiated myocyte-containing cardiac organoids are incubated in an appropriate cardiomyocyte maintenance medium.

A plurality of culture media suitable for maintaining cardiomyocytes in culture are already known in the art, including a plurality of commercial media for maintaining cardiomyocytes in culture.

The skilled artisan may notably refer to Zhu et al. (2011, Methods Mol Biol, Vol. 767:419-431).

In preferred embodiments, step e) is performed by culturing the mammal differentiated myocyte-containing cardiac organoids obtained at step d) in the serum-free cardiomyocyte maintenance medium commercialized by company ThermoFisher Scientific as “PSC Cardiomyocyte Maintenance Medium”.). For the qualitative and quantitative constitution of the cardiomyocyte maintenance medium, the skilled artisan may refer to the article by Blurridge et al. (2014, Nature Methods, Vol. 11:855-860)

Beating cardiac organoids, i.e. comprising contractile cardiomyocytes, are readily obtained after a culture time period ranging from about 5 to about 10 days, at step e) of the method, such as from about 7 to about 10 days.

As it is illustrated in the examples herein, the beating cardiac organoids may be viably cultured for a long time period at step e), including for a time period of 120 days or more, which encompasses a time period ranging from 5 to 200 days.

### Cardiac Organoids Obtainable by the Disclosed Method

According to another aspect, the present disclosure relates to cardiac organoids obtainable by the disclosed method, which means obtainable by performing step a) to step e) of the disclosed method.

As it is shown in the examples herein, the cardiac organoids obtained at step e) of the disclosed method, which can be subject to long term culture at the said step e), have a plurality of features that qualify them as relevant in vitro test models; both of healthy cardiac organs and of cardiac organs from subjects affected with a cardiac disorder or disease.

The cardiac organoids obtained by the disclosed method express cardiac-specific biomarkers, such as Sarcomeric Alpha Actinin (SA), MLC-2A and MLC-2V. The cardiac organoids also possess relevant ultrastructural features, such as myofibrils.

First, the cardiac organoids obtained by the disclosed method are of a spheric shape with heart chamber-like structures.

As shown in the examples, the mean diameter of the cardiac organoids increases with the time of culture, at step e) of the disclosed method. Their mean diameter can reach around 400 μm or more after 14 days of culture, at step e) of the disclosed method, and even a mean diameter of 2000 μm or more, when derived from IPSCs originating from c-met subjects.

After 30-50 days of culture, control organoids continue to increase in size (500 μm-2000 μm) whereas c-met organoids can increase from 1000 μm to 5000 μm.

Importantly, as it is shown in the examples, the cardiac organoids obtained by the disclosed method express both atrium-like chamber markers, such as MLC-2A and ventricular-like chamber markers, such as MLC-2V, which markers are detectable in distinct regions of the cardiac organoids, which denote an organ-like organization pattern.

Also importantly, the cardiac organoids obtained by the disclosed method possess a capillary structure, with the detectable presence of numerous capillaries therein.

As shown in the examples herein, a mean number of 800 capillaries per organoid have been measured in cardiac organoids obtained by the disclosed method. Also, in these cardiac organoids, a mean total length of the capillaries may reach about 15000 μm have been measured. The presence of capillaries, thus of endothelial cells, in the organoids obtained by the disclosed method is all the more surprising that the said cardiac organoids stem from the monoculture and differentiation of a unique cell type, namely a single cell line of induced pluripotent stem cells. Indeed, for cardiac organoids obtained, according to the disclosed method, from iPSCs originating from mature cells previously sampled from patients affected with some diseases, like cardiac hypertrophy, those cardiac organoids have typically an increased size.

It is also shown herein that the cardiac organoids obtained by the disclosed method possess contractile properties, i.e. the said cardiac organoids are beating cardiac organoids, which is illustrated by the contractile properties of the cardiomyocytes contained therein.

Consequently, according to the applicant's knowledge, cardiac organoids obtainable by the method disclosed herein possess unique features that distinguish them from previously described cardiac organoids.

The present disclosure also relates to cardiac organoids having one or more of the following features:
the presence of both atrial-like and ventricular-like cell pattern,
the presence of both atrial chamber-like and ventricular chamber-like structures, as it is easily visualized, for instance, by immunochemistry
the presence of a network of vascular capillaries, and
the presence of contractile properties, as it is easily determined, for instance, through the use of a microscope as well as by using electrophysiological methods known in the art.

For the sake of clarity, the cardiac organoids obtained by the disclosed method, beyond comprising atrial-like cells and ventricular-like cells, are also structurally organized so as to exhibit both atrial chamber-like and ventricular chamber-like structures

### Uses

Cardiac organoids obtainable by the method disclosed herein consist of suitable in vitro models reproducing the complexity of the mammal cardiac organ, which includes the human cardiac organ.

Consequently, cardiac organoids obtainable by the disclosed method can be used for various purposes, including for research on the cardiac organ physiology and for more direct medical purpose, such as for screening for substances for their in vitro qualification as candidate active agents aimed at preventing or treating heart disorders of diseases.

The present disclosure relates to the in vitro use of a cardiac organoid obtainable by the disclosed method for testing the activity of a substance endowed with physiological effects.

In preferred embodiments, the substance endowed with physiological effects consists of a drug candidate.

As it is shown in the examples herein, the method disclosed herein allows obtaining cardiac organoids by using either (i) iPSCs cells derived from a healthy mammal, including a mammal who is not affected with a heart disorder or disease or (ii) iPSCs cells derived from a mammal that is affected with a heart disease or disorder.

Notably, the inventors have shown herein that the disclosed method allows obtaining cardiac organoids that recapitulate the heart disorder or disease affecting the subject from which the iPSCs provided at step a) originated. More precisely, it is shown in the examples herein that providing at step a) iPSCs that have been generated from an adult cell sample originating from a subject having a mutated c-met and expressing a cardiac hypertrophy, allowed obtaining with the disclosed method cardiac organoids recapitulating a plurality of physiological parameters of an hypertrophic cardiac organ. Also shown in the examples is the testing of a heart hypertrophy candidate active substance.

Thus, according to some aspects, the present disclosure pertains to the in vitro use of a cardiac organoid obtainable by the method disclosed herein, wherein the mammal iPSCs derive from the programmation of a sample of differentiated human cells originating from a subject affected with a cardiac hypertrophy, for testing the activity of a drag candidate against cardiac hypertrophy. In some embodiments, the differentiated human cells having a mutation on the c-met gene.

The present disclosure also pertains to an in vitro method for the screening of a substance for preventing or treating a cardiac disorder or disease, comprising the steps of:
a) providing a cardiac organoid as disclosed herein,
b) bringing into contact a candidate substance with the cardiac organoid provided at step a),
c) determining if the said candidate substance causes a change in one or more parameter that is indicative of a therapeutic effect
d) selecting the candidate substance when a change in one or more parameter that is indicative of a therapeutic effect has been determined at step c).

Effect of cell function can be assessed using any standard assay to observe phenotype or activity of cardiomyocytes, such as marker expression, receptor binding, contractile activity, or electrophysiology—either in cell culture or in vivo. Pharmaceutical candidates can also be tested for their effect on contractile activity—such as whether they increase or decrease the extent or frequency of contraction. Where an effect is observed, the concentration of the compound can be titrated to determine the median effective dose (ED₅₀).

Cardiac disorders or diseases encompass heart rhythm disorders, cardiomyopathies, congenital heart diseases, structural heart diseases, such as cardiac hypertrophy, and heart failure.

The present disclosure also relates to the testing of pharmaceutical compounds for their effect on cardiac muscle tissue maintenance or repair. Screening may be done either because the compound is designed to have a pharmacological effect on the cells, or because a compound designed to have effects elsewhere may have unintended side effects on cells of this tissue type.

Consequently, the present disclosure also pertains to an in vitro method for the testing the toxicity of a substance for the cardiac organ, comprising the steps of:
a) providing a cardiac organoid as disclosed herein,
b) bringing into contact a substance to be tested with the cardiac organoid provided at step a),
c) determining if the said candidate substance causes cytotoxicity in the cardiac organoid.

Cytotoxicity can be determined in the first instance by the effect on cell viability, survival, morphology, and the expression of certain markers and receptors.

The present disclosure is further illustrated, without in any way being limited to, the following examples.

### EXAMPLES

### a. Materials and Methods

### A.1. Reagents and Resources

| REAGENT or RESOURCE | SOURCE | IDENTIFIER |
|---|---|---|
| Antibodies | | |
| Sarcomeric Alpha Actinin | Abcam, EP2529Y | ab68167 |
| Phospho-Met (Tyr1234/1235) | Cell Signaling Technologies | 3077S |
| Phospho-STAT3 (Tyr705) | Cell Signaling Technologies | 4113S |
| MLC-2A | synaptic systems | 311 011 |
| MLC-2V | synaptic systems | 310 003 |
| Cardiac troponin T | Abcam | ab91605 |
| CD31 | Abcam | ab24590 |
| Caspase-3 | Cell Signaling Technologies | 9662S |
| ProBNP | Abcam | ab239519 |
| DAPI | Sigma-aldrich | D9542 |
| Chemicals, Peptides, and Recombinant | | |
| Proteins | | |
| Essential 8 basal medium | Thermo Fisher Scientific | A1516901 |
| Essential 8 supplement | Thermo Fisher Scientific | A1517101 |
| Geltrex ® LDEV-Free Reduced Growth | Thermo Fisher Scientific | A1413202 |
| Factor Basement Membrane Matrix | | |
| PSC Cardiomyocyte Differentiation Kit | Thermo Fisher Scientific | A2921201 |
| Cardiomyocyte Maintenance Medium | | |
| Cardiomyocyte Differentiation Medium A | Thermo Fisher Scientific | A2921201 |
| Cardiomyocyte Differentiation Medium B | Thermo Fisher Scientific | A2921201 |
| Experimental Models: Cell Lines | | |
| Human iPSC: PB33 | Human | — |
| Human iPSC: PB56 | Human | — |
| Cisplatin | Sigma-aldrich | P4394 |
| HGF | R&D Systems | 294-HG |
| SU11247 | Sigma-aldrich | S9820 |
| Osmium tetroxide solution | Sigma-aldrich | 75632 |
| Glutaraldehyde grade I | Sigma-aldrich | G5882 |
| Software and Algorithms | | |
| ImageJ | | |
| Imaris | | |
| LAS AF | | |
| Myocyter 1.0 | | |

A.2. Generation and Culture of iPSC

The iPSC lines used in this study were previously described (Hwang et al., 2019, Int J Mol Sci, Vol. 20:doi:10.3390/ijms20194867). Briefly, iPSC culture was performed according to two different procedures: in the presence of feeders or in feeder-free conditions. The feeder cultures were performed on Mitomycin C-treated MEF layers as described (40) with passaging every 7 days using 1 mg/mL collagenase IV in DMEM/F12 (Thermo Fisher Scientific). Feeder-free cultures were performed on Geltrex™ (Thermo Fisher Scientific) in essential 8 medium (Thermo Fisher Scientific) and 1% penicillin/streptomycin with passaging every 3-4 days using in DPBS (Thermo Fisher Scientific) supplemented with 0.5 mM EDTA (Thermo Fisher Scientific) and 1.8 mg/L NaCl (Sigma).

### A.3. Cell Culture and Aggregate Generation

Undifferentiated human iPSC and c-met-mutated iPSC were maintained on Geltrex™ (Thermo Fisher Scientific) coated flat culture dish in E8 media (Thermo Fisher Scientific) according to manufacturer's guidelines. Colonies were manually harvested until 60-80% confluence. Then, cells were collected and dissociated into single cells using EDTA before proceeding to the formation of aggregates using either ultra-low attachment 24-well or 96-well plates in static (for comparison) or dynamic (this disclosure) conditions, respectively, as described below.

A.4. Matrix-Free iPSC Aggregation-Mediated 3D-Cultures for Induction of Cardiac Organoids

To accelerate the induction of aggregation procedure for cardiac differentiation, single cell suspensions after detaching iPSC by EDTA were put onto ultra-low attachment 24-well or 96-well plate (Corning, Inc) at a concentration of 1×10⁵ or 1×10⁴/well respectively. In the 96-well plate cell culture, dynamic shaking was performed on an orbital shaker rotating at 70 rpm for 1 h allowing the initiation of the formation of cell aggregates, followed by static culture for 24 hours. This procedure allowed formation of well-developed aggregates in both type of wells after 1 day in E8 medium (Stem Cell Technologies). As shown in FIG. 1D, cells were then cultured in cardiac induction media A (Thermo Fisher Scientific) for 1 day, switched to cardiac induction medium B (Thermo Fisher Scientific) and cultured for an additional day. At day +3; cells were switched to cardiac maintenance medium C (Thermo Fisher Scientific), with until day 120 with medium changes performed every 3-4 days. Using this protocol, beating cardiac organoids were found to appear 7-10 days and they exhibited a persistent beating behavior up to day +120.

### A.5. Whole-Mount Immunostaining of 3D Cardiac Organoids

Cardiac organoids cultured on 24 or 96-well culture plates were washed with phosphate-buffered saline (PBS), fixed with 4% paraformaldehyde in PBS for 60 min, permeabilized with 0.2% Triton X-100 (Sigma) in PBS and blocked 10% serum. Primary antibodies were diluted in PBS 10% serum at the following concentrations: phospho-Met; 1:100 (Cell Signaling Technologies), phospho-STAT3; 1:200 (Cell Signaling Technologies), Sarcomeric Alpha Actinin; 1:200 (Abcam), MLC-2A; 1:100 (Synaptic Systems), MLC-2V; 1:100 (Synaptic Systems), Cardiac troponin T; 1:100 (Abcam), CD31; 1:100 (Abcam), Caspase-3; 1:100 (Cell Signaling Technologies), ProBNP; 1:100 (Abcam), and then washed three times in PBS. The samples were incubated with secondary antibodies in antibody dilution buffer, then washed in PBS. Nuclei were labeled with DAPI mounting medium. The negative control performed a secondary antibody only (data not shown). Visualization and capture were realized with a Leica confocal microscope and LAS AF software.

### A.6. Transmission Electron Microscopy (TEM)

Aggregates were gently centrifuged and pelleted before the TEM process as follows. The cells were fixed in 2.5% glutaraldehyde in phosphate-buffered saline (PBS) for 1 h at 4° C., washed in PBS, and fixed in 1% osmium tetroxide in PBS for 1 h. They were dehydrated in ascending series of graded ethyl alcohols, then in acetone. Each sample was infiltrated with the resin before being embedded in epoxy resin and polymerized for 72 h at 60° C. Semi-thin sections of about 0.5 to 1 μm were obtained and colored with Toluidine blue before being examined via a light microscope with an associated digital camera, hooked to a computer for image processing and editing (Leica DC 300). Ultra thin sections of about 60/90 nm were contrasted with heavy metals (uranyl acetate and lead citrate) and were examined using a Jeol 1010 transmission electron microscope at an accelerated voltage of 80 kV. Images were photographed on digital images Gatan Digital Micrograph: brure Erlangen 500w: camera and edited by Microsoft Power Point.

### A.7. Organoid Image Rendering and Analysis

3D image reconstructions were used to analyze microvessels, muscle fibers and rotating movies were performed with Leica confocal microscope images, LAS AF software, ImageJ and Imaris software.

### A.8. Cobalt Chloride-Mimicked Hypoxia

Cardiac organoids cultured for 3-4 months in 96 wells were transferred randomly to XFp mini plates (1-6 organoids/well, 300 to 1100 μm in diameter) (Seahorse, Agilent). The cardiac culture medium was treated with 200 μM CoCl2 (sigma) concentration for 24 hours. To evaluate the effects of HGF stimulation and c-met inhibition we have used respectively HGF at 50 ng/ml (R&D Systems) and SU11274 at 5 M (Sigma) under hypoxia.

### A.9. Measurement of Oxygen Consumption Rate (OCR) and Extracellular Acidification Rate (ECAR)

OCR, ECAR and energy phenotype test were all measured using a Seahorse XFp analyzer (Seahorse, Agilent). On the day of testing, cells were incubated in XF assay medium for 1 hr at 37° C. in non-CO2 incubator before measurement. Cell energy phenotype kit was used with port A containing the FCCP/oligomycin stressor mix at 2.0/1.0 μM (final well concentration). OCR and ECAR were normalized to the cell numbers of each wells. Cells were stained with DAPI and calculated from Leica confocal microscope images.

### A.10. Cardiac Organoid Toxicity Test

Organoids in identically plated wells of a 24-well plate were treated with a single concentration of cisplatin (500 μmol/well) for 30 min. We measured spontaneous contractions by a Leica confocal microscope.

### A.11. Cardiomyocyte Contractility

Myocyter (Grune et al., 2019, J Sci Rep, Vol. 9:15112) and ImageJ were used to measure the contractile behavior of the myocardium. Myocyter can characterize cardiomyocyte contractility according to changes in pixel intensity of recorded cardiomyocytes. We calculated contraction amplitude and frequency in both groups.

### A.12. Statistical Analysis

The data are generally presented as the means of number of replicates±SD. All data are graphed and analyzed for significance using a Student's t-test.

### A.13. Transcriptome Analysis of Cardiac Organoids

This analysis was performed in beating cardiac organoids collected between days +40 and +50. Total RNA from cardiac organoids (wild-type versus c-met mutated) was extracted in duplicate to be processed to whole transcriptome analysis. After probe synthesis Clariom S human arrays (Thermo Fisher) were hybridized and scanned on Affymetrix platform. Raw transcriptome data were normalized by Robust Microarray Analysis (RMA) (Irizarry et al., 2003, Nucleic Acids Res, Vol. 31:e15). Differential expressed gene (DEG) analysis was performed with LIMMA algorithm with limma R-package version 3.44.3 (Ritchie et al., 2015, Nucleic Acids Res, Vol. 43:e47). Expression heatmap (classification with Euclidean distances and ward.D2 method) was performed with pheatmap R-package version 1.0.12 on up regulated genes in c-met cardiac organoid (log fold change>2 & False Discovery Rate q-values<0.005). Functional enrichment analysis was performed with the web application Toppgene (Chen et al., 2009, Nucleic Acids Res, Vol. 37:W305-311) Cardiac functional enriched networks were drawn with Cytoscape standalone software version 3.6.0 (Cline et al., Nat Protoc, Vol. 2:2366-2382). Bioinformatics analyses were performed in R software environment version 4.0.2.

### Example 1: Characterization of iPSC

Control iPSC and c-met-mutated iPSC described previously (Hwang et al., 2019, Int J Mol Sci, Vol. 20:doi:10.3390/ijms20194867) were maintained on Geltrex coated plates. As reported, they exhibited pluripotency markers and the presence of c-met mutation harboring p. (Val1238Ile) was confirmed by Sanger sequencing. They were also characterized with regard to the expression of phospho-Met (Tyr1234/1235) and phospho-STAT3 (Tyr705) respectively, by immunostaining (FIG. 1A). To investigate the possibility of induction of cardiac organogenesis, we first developed a matrix-free aggregation-mediated strategy and we have generated control iPSC aggregates and c-met-mutated iPSC aggregates in ultra-low attachment plates. These cells have then been used to generate beating cardiac organoids in 3D conditions.

### Example 2: Development of Conditions Leading Beating Cardiac Organoids

Control and c-met-mutated iPSC aggregates cultured in ultra-low attachment plates were further cultured either in static or using rotational conditions, for comparison purposes, and then allowing cell aggregation in static culture conditions was performed (FIG. 1B). In order to find the optimal cardiac differentiation conditions, several timepoints of cultures in the presence of differentiation media were tested. The best timing to start cardiac differentiation in the presence of specific media was found to be after 1 day of culture in the presence of E8 medium (FIG. 1B). As shown in FIG. 1B, cells were cultured in media A for 1 day and media B for an additional day, before switching to cardiac maintenance medium from day+3. (FIG. 1B). These experiments showed the feasibility of obtaining beating organoids in both types of iPSC when performing a rotational step (dynamic culture conditions). During these steps, both control and c-met-mutated iPSC aggregates led to the generation of structures in the form of spheres or chambers, but c-met-mutated iPSC-derived (c-met) cardiac organoids reached a gigantic size. Interestingly, at day +30-50 of cultures, in the c-met group (total organoids; n=31, three experiments), 29% of organoids exhibited a diameter of more than 2000 μm whereas in the control group (total organoids; n=73, three experiments), none of the organoids reached this size. Indeed, as seen in FIG. 2, at day +30-50, the size of c-met cardiac organoids (most population was in the below of 3000 μm) was much larger than that of control cardiac organoids (most population was in the below of 1500 μm) and this was reproducible in several experiments.

### Example 3: Characterization of Cardiac Organoids

To confirm the properties of cardiac organoids, we first used immunostaining using Sarcomeric Alpha Actinin (SA). As it has been observed, SA signals were strongly detected in both control (SA+ cells intensity; 0.57±0.10) and c-met cardiac organoids (SA+ cells intensity; 0.61±0.18). Next, immunostaining confirmed expression of phospho-STAT3 (Tyr705), known as one of the activated c-met downstream signaling proteins in both control and c-met cardiac organoids. These stainings were completed by the evaluation of the expression of phospho-Met (Tyr1234/1235) and phospho-STAT3 (Tyr705) which were found to be highly expressed in c-met cardiac organoids (pMet+ cells intensity; 0.58±0.17, pSTAT3+ cells intensity; 0.40±0.04) (FIG. 3). In contrast, control iPSC-derived cardiac organoids showed weak phospho-Met and phospho-STAT3 signals (pMet+ cells intensity; 0.09±0.04, pSTAT3+ cells intensity; 0.10±0.06).

### Example 4: Ultrastructure Analysis

We next wished to characterize cardiac organoids in both groups by ultrastructure analysis using transmission electron microscopy (TEM). As it has been observed, sarcomere and cell-to-cell junctions were present in both groups at 14 days. Interestingly, myofibrils detected in the control group were found to be disorganized at day +30 whereas well-organized myofibrils with atrial-specific granules (SG) were detectable in c-met group.

### Example 5: Chamber-Like Structure Formation and Cardiac Organoid Vascularization

To investigate whether our 3D culture can generate ventricular- and atrium-like chambers in cardiac organoids, we used myosin light chain 2 ventricle (MLC-2V) and myosin light chain 2 atrium (MLC-2A) antibodies, which are well known as ventricular and atrium muscle markers (FIG. 4A, 4B). MLC-2V, was strongly expressed in all cardiac organoids in both groups. (n=3, FIG. 4B, c-met group, MLC-2V+/DAPI+ cells; 64.71%+2.03, control group, MLC-2V+/DAPI+ cells; 71.69%±2.61). MLC-2A, an atrium-like chamber marker, was detected in distinct regions in both groups. Interestingly, there were more MLC-2A expressing cells in c-met group (n=3, FIG. 4B, c-met group, MLC-2A+/DAPI+ cells; 23.91%±3.19) as compared to control group (n=3, FIG. 4B, control group, MLC-2A+/DAPI+ cells; 9.71%±3.81). Moreover, as seen FIG. 4C, diameters of ventricular-like region and atrium-like region were significantly higher in c-met group (diameter of ventricular; 909.30±213.86 μm, diameter of atrium; 292.95±31.81 μm) as compared to control group (diameter of ventricular; 584.69±70.27 μm, diameter of atrium; 82.81±32.80 μm). Computer image analyses revealed that the expressions of MLC-2A and MLC-2V were detectable in distinct regions of the organoids, suggesting an organ-like organizational pattern. The immunostaining results suggested that our 3D culture system effectively induced the formation of ventricular- and atrium-like chambers.

We next asked whether these structures can be accompanied by the generation of blood vessels. As can be seen in FIG. 4D and FIG. 5 (5A, 5B), immunostaining and optimized computational vascular image analysis using anti-CD31 staining confirmed that our 3D culture system induced capillary formation in both groups. In particular, organoids from c-met group shows the presence of more numerous and longer capillaries (n=3, FIG. 4D, number of capillaries/organoids; 1597.0±82.34, length of capillaries/organoids; 27880.79±2663.17 μm) than control (n=3, FIG. 4D, number of capillaries/organoids; 832.75±416.79, length of capillaries/organoids; 14823.15±7976.91 μm). Taken together, these findings suggest that our 3D culture system induced ventricular- and atrium-like chambers and along with capillaries in the cardiac organoids. Notably, c-met cardiac organoids exhibited significantly enhanced positivity using ventricular, atrium and capillary markers as compared to control organoids.

### Example 6: Analysis of c-Met-iPSC-Derived Hypertrophic Cardiac Organoids

For these analyses we used a cardiac troponin-T antibody for measuring of cardiac muscle size and a proBNP antibody for mimicking a potential heart dysfunction. As seen FIG. 6A, 6B and FIG. 7A, 7B, the diameters of cardiac troponin T+fibers in c-met cardiac organoids (n=3, FIG. 6A and FIG. 7A, 7B, average diameter; 5.58±2.01 μm) were greater than control cardiac organoids (n=3, FIG. 6A and and FIG. 7A, 7B, average diameter; 3.96±1.12 μm). In particular, c-met group shows the presence of larger and thicker fibers (n=3, FIG. 6A, volume of fibers/organoids; 75660.16±25196.48 μm³) than control group (n=3, FIG. 6A, volume of fibers/organoids; 22087.69±16003.17 μm³). proBNP expression test was then evaluated in organoids cultured in 96 wells for 3-4 months. As seen FIG. 6B, proBNP and caspase-3 expressions were strongly detected in c-met cardiac organoids (n=4, FIG. 6B, proBNP+ cells intensity; 0.73±0.15, caspase-3+ cells intensity; 0.23±0.09). In contrast, control cardiac organoids showed weak proBNP and caspase-3 signals (n=5, FIG. 6B, proBNP+ cells intensity; 0.38±0.11, caspase-3+ cells intensity; 0.09±0.06). Interestingly, hypertrophic heart development can be predicted through significantly higher expression of proBNP and cardiac troponin T markers in c-met group than in the control group.

### Example 7: Hypoxia Resistance

It has been previously reported that c-met has a beneficial effect on cardiac defense against apoptosis, contributing to cardiomyocyte protection in myocardial ischemia (Nakamura et al., 2000, J Clin Invest, Vol. 106(12):1511-1519). To demonstrate the potential effect of c-met expression in hypoxia resistance, we have established an organoid culture methodology based in cobalt-induced hypoxia. After 24 h of hypoxia treatment, we performed immunostaining with proBNP, caspase-3 antibodies, and measured OCR and ECAR with an energy phenotype kit. As can be seen FIG. 6C, caspase-3 expression was strongly detected in control cardiac organoids treated in hypoxia-inducing conditions (n=5, caspase-3+ cells intensity; 0.61±0.11). In contrast, c-met iPSC-derived cardiac organoids showed weak caspase-3 signals in the same conditions (n=8, caspase-3+ cells intensity; 0.34±0.16). ProBNP was expressed similarly in both groups. Interestingly, after hypoxia treatment, the control group showed high expression of proBNP (n=5, proBNP+ cells intensity; 0.78±0.16), while c-met group (n=8, proBNP+ cells intensity; 0.73±1.4) showed similar expression to normoxia (FIG. 6C). We then wished to test the mitochondrial function in cardiac organoids in the same hypoxia-inducing conditions for 24 h followed by the analysis of energy phenotype, OCR and ECAR of cardiac organoids using a Seahorse Analyzer (FIG. 8). To this end, we randomly selected organoids (average organoid diameters: control; 985.5±526.8 μm, c-met; 1129.5±442.4 μm) cultured in 96 wells for 3-4 months. When comparing the energy phenotype in baseline activity to control group and c-met group, it was observed that control cardiac organoids are more energetic with increased glycolytic activity, as compared to c-met cardiac organoids are more quiescent, they have less glycolysis (FIGS. 8, A, D, G, J and N). On the other hand, after hypoxia treatment, the energy phenotype and ECAR results show two distinct effects of c-met. First, after hypoxia treatment, c-met cardiac organoids are more energetic than control. Moreover, glycolysis activity in control group decreased as compared to c-met group (FIGS. 8, B, E, H, K and M). Second, c-met cardiac organoids exposed to hypoxia with c-met inhibitor; SU11274 were found to exhibit a significantly decreased glycolysis activity at the baseline as compared with HGF-treated control (FIGS. 8, C, F, I, L and O). Taken together, these results suggest that under hypoxia, c-met cardiac organoids are more energetic than control organoids and that c-met maintains the mitochondrial function of cardiomyocytes.

### Example 8: Contractility and Drug Test of Cardiac Organoids

We next analyzed control and c-met cardiac organoids amplitude and frequency, as well as contractions recorded by confocal microscopy. We randomly selected three organoids from each group with defined diameters (average organoid diameters: control; 359.3±39.1 μm, c-met; 1037.3±59.9 μm) cultured in 24 wells at 24 days. The contractility was studied using the recently developed myocyter macro and imageJ software (FIG. 9A). As can be seen from the FIG. 9B, 9C, 9D, 9E, it was found that the relative amplitude and frequency of contractions observed in c-met cardiac organoids were increased compared to the control. Next, to test whether our organoids could be used to study cardiac toxicity in vitro, we used cisplatin (Sigma) with a well-described cardiotoxicity behavior (El-Awady et al., 2011, Eur J Pharmacol, Vol. 650:335-341) at a concentration of 500 μmol for 30 min as a modified method previously described (Forsythe et al., 2018, Front Public Health, Vol. 6:doi:10.3389/pubh.2018.00103). As shown in FIG. 9F, 9G, 9H, cardiac organoid relative amplitude was decreased as soon as after 30 min of cisplatin exposure (FIG. 9F) in both groups, Moreover, contraction frequency was significantly decreased in c-met cardiac organoids (FIG. 9E). On the other hand, control cardiac organoid frequency was not found to be clearly reduced. To further analyze these events, we used various thresholds (at 10, 50, and 90%) distinguish amplitude (FIG. 9F), contraction (FIG. 9G), and relaxation time (FIG. 9H). These times were decreased according to thresholds in both groups. Interestingly, after drug treatment, the amplitude, contraction, relaxation time were increased at 10% and 50% thresholds only in control group. These data showed that decreasing of relative amplitude, frequency, amplitude, contraction and relaxation time are different between control cardiac organoids and c-met cardiac organoids. It could be used to test the drug toxicity for personalized medicine.

### Example 9: Transcriptome Analyses

A whole transcriptome analysis was performed in wild-type and c-met (duplicates) cardiac organoids derived from human iPSC. Differentially expressed gene analysis realized by LIMMA algorithm between the two experimental conditions (wild-type organoid as reference) identified mainly a repressed signature by the c-met mutation. Genes found to be up regulated (n=111, log fold change>2 & False Discovery Rate q-values<0.005) allowed to discriminate the two experimental conditions. Functional enrichment with up regulated genes in c-met cardiac organoids on Gene Ontology Biological Process database identified implication of functionalities implicated in cardiac development (FIG. 10A, 10B). Functional enrichment performed with genes up regulated by c-met mutation in cardiac organoids on mouse phenotype database allowed to highlight several cardiac abnormalities (FIG. 10C) and especially some genes were found enriched in cardiac hypertrophy conditions (FIG. 10D). All together these results suggested that c-met mutation induced the up regulation of an expression profile in cardiac organoids that is known to be implicated in cardiac development but also in cardiac hypertrophy.

### Discussion of the Results Disclosed in the Examples

We describe in this work a method for generating functional cardiac organoids and its potential application to pathology by using the example of organoids derived from a c-met-mutated iPSC. There are several protocols described previously for the generation of cardiac organoids from human ESC or iPSC. In comparison with these protocols, the method described here shows the ability to reduce the culture time as compared to previous protocols (Shkumatov et al., 2014, PLOS ONE, Vol. 9:doi:10.1371/journal.pone.0094764; Hoang et al., 2018, Nat Protoc, Vol. 13:723-737). Indeed, we show here that it is possible to produce beating cardiac organoids already within 7-10 days without additional physical chambers or the use of a matrix such as hydrogel. The technique implicates, in either static or dynamic conditions the generation of iPSC aggregates formed by culture in the presence of cardiac differentiation media (such as differentiation media A and B illustrated in the examples). We report the reproducibility of this technique (over five experiments) and its potential application to the engineering of an organoid from a c-met-mutated iPSC that could be used for drug testing purposes. Transcriptome analyses revealed that genes involved in cardiac development were strongly expressed in the c-met group compared to the control group. Interestingly, genes involved in HCM such as MYL2, ACTC1, TNNC1, CSRP3, TTN and PLN (Li et al., 2019, J Clin Med, Vol. 8: doi:10.3390/jcm8040520) were found to be highly upregulated in c-met iPSC-derived cardiac organoids as compared to control. Previous reports highlighted the well-established role of these genes reported in on HCM (Ingles et al., 2019, Circ Genomic Precis Med, Vol. 12: e002460). Importantly, two of the genes identified in our work (MYL2 and ACTC1) were listed amongst the 8 genes definitively associated with familial HCM syndromes in humans (Ingles et al., 2019—Supra). The model that we describe herein is of major interest for early drug development in these diseases as well as testing the validation of these syndromic genes in experimental conditions.

We report here that a matrix-free and aggregation-mediated 3D culture system can thus generate beating human cardiac organoids derived from both control and c-met-mutated human iPSC. The differences that we detected in the kinetics of heart generation and in transcriptome suggest that c-met plays a role on heart diseases in humans. Indeed, the HGF/c-Met signaling pathway plays an important role in the development of mouse cardiac diseases such as hyperplastic and hypertrophic growth. These results suggest the possibility of modeling cardiac diseases in organoids based on c-met-mutated iPSC. On the other hand, we confirmed that c-met can contribute to reducing cellular apoptosis and maintaining mitochondrial respiration and glycolysis activity after hypoxia as compared to controls. The c-met-mutated cardiac organoids strongly express phospho-Met and phospho-STAT3 as compared to control cardiac organoids. Notably, STAT3, which is one of the downstream signaling pathways of activated c-met is also known to play an important cardioprotective effect by upregulation anti-apoptotic and angiogenic genes (Osugi et al., 2002, J Biol Chem, Vol. 277:6676-6681; Hilfiker et al., 2004, Circ Res, Vol. 95:187-195). Moreover STAT3 has been proposed to perform a protective function in mitochondria and cardiac protection against a variety of heart pathologies such as myocardial ischemia and hypertrophy (Harbous et al., 2019, Front Cardiovasc Med, Vol. 6:doi:10.3389/fcvm.2019.00150). It is noteworthy that STAT3, in contrast to those described above, has multiple roles in the heart. For cardiomyocyte pathology, overexpression STAT3 is known to cause cardiac hypertrophy in mouse (Kumisada et al., 2000, Proc Natl Acad Sci USA, Vol. 97:315-319). In c-met-mutated iPSC, we confirmed the generation of giant-diameter cardiac organoids and the c-met group had larger and thicker cardiac fibers than the control group. Interestingly, we showed that phospho-Met overexpression enhances atrial, ventricular and capillary cell formation as compared to control.

In summary, the feasibility of generation of cardiac organoids with atrial-, ventricular-like chambers with well-developed vascular networks through in vitro self-assembly using human iPSC, is fully demonstrated herein. This model shows a morphological improvement over previously developed cardiac organoid models. In particular, with this method, we demonstrated for the first time that c-met iPSC-derived 3D cardiac organoids that could represent an important model of cardiac hypertrophy. Previous studies of the pathology of iPSC-derived cardiac hypertrophy have used in vitro 2D monolayer (Wu et al., 2019, Eur Heart J, Vol. 40:3685-3695) or engineered heart tissues (Prondzynski et al., 2019, EMBO Mol Med: Vol. 11(12):e11115), which have failed to recapitulate cardiac-related architectures. On the other hand, c-met iPSC-derived cardiac hypertrophy organoids have morphologically improved by forming giant chambers with well-developed vascular networks.

We show that the c-met-mutated cardiac organoid model express several genes with definitive roles established in HCM. The robust expression of proBNP, a strong prognostic indicator for heart failure makes this methodology as a potential use for further modeling of heart diseases.

Overall, cardiac organoids obtained by the matrix-free method disclosed herein has the potential to contribute to the study of cardiac pathology and drug toxicity studies of structural abnormalities. In addition, such cardiac organoids can be cultured for more than four months, it provides a relevant model for long-term drug toxicity testing instead of animal experiments.

Finally, matrix-free method for producing a bioengineered mammal induced pluripotent stem cells-derived (IPSCs-derived) cardiac organoid that we describe here is of an outmost interest for the future development of personalized medicine in cardiology.

1. An in vitro matrix-free and feeder-free method for producing a cardiac organoid comprising the steps of:
a) providing mammal iPSCs,
b) culturing the mammal iPSCs provided at step a) under dynamic culture conditions during a time period ranging from 30 minutes to 3 hours, whereby formation of iPSCs cell aggregates is initiated,
c) culturing under static culture conditions the cell aggregates under formation obtained at step b), during a time period ranging from 12 hours to 48 hours, whereby iPSCs cell aggregates are obtained,
d) incubating the cell aggregates obtained at step c) in a cardiomyocyte differentiation medium, whereby a differentiated myocyte-containing organoid is obtained, and
e) culturing the differentiated myocyte-containing organoid obtained at step d), whereby a cardiac organoid is obtained.

2. The method according to claim 1, wherein the mammal iPSCs provided at step a) consist of human iPSCs.

3. The method according to claim 1, wherein step b) is performed during a time period of about 1 hour.

4. The method according to claim 1, wherein step d) is performed during a time period ranging from 1 to 5 days.

5. The method according to claim 1, wherein step d) is performed by incubating the iPSCs aggregates obtained at step c) successively in more than one cardiomyocyte differentiation medium.

6. The method according to claim 5, wherein step d) is performed by incubating the iPSCs aggregates obtained at step c) successively (i) in the Cardiomyocyte Differentiation Medium A and (ii) the Cardiomiocyte Differentiation medium B comprised in the commercial PSC Cardiomyocyte Differentiation Kit marketed by Thermofischer Scientific company.

7. The method according to claim 1, wherein step e) of culturing the differentiated myocyte-containing organoid obtained at step d) has a duration of 5 days or more, preferably of 7 days or more.

8. The method according to claim 7, wherein step e) is performed during a time period ranging from 5 to 200 days.

9. The method according to claim 1, wherein the mammal iPSCs provided at step a) derive from the programmation of a sample of differentiated human cells.

10. The method according to claim 1, wherein the mammal iPSCs provided at step a) derive from the programmation of a sample of differentiated human cells originating from a subject affected with a cardiac disease.

11. (canceled)

12. A method for testing the activity of a substance endowed with physiological effects comprising the steps of:
implementing the method according to any one of claims 1 to 10 so as to obtain a cardiac organoid,
using said cardiac organoid in vitro for testing the activity of the substance endowed with physiological effects.

13. The method according to claim 12, wherein the substance endowed with physiological effects consists of a drug candidate.

14. A method for testing the activity of a drug candidate against cardiac hypertrophy comprising the steps of:
implementing the method according to claim 10 wherein the cardiac disease is cardiac hypertrophy, so as to obtain a cardiac organoid,
using said cardiac organoid in vitro for testing the activity of the drug candidate against cardiac hypertrophy.

15. The method according to claim 14, wherein in the implementing step the differentiated human cells have a mutation on the c-met gene.

16. A cardiac organoid having one or more of the following features:
the presence of both atrial-like and ventricular-like cell pattern,
the presence of both atrial chamber-like and ventricular chamber-like structures
the presence of a network of vascular capillaries, and
the presence of contractile properties.

17. The cardiac organoid according to claim 16, which is structurally organized so as to exhibit both atrial chamber-like and ventricular chamber-like structures.
